(19) United States
(12) Patent Application Publication
(10) Pub. No.: US 2007/0293458 A1
(43) Pub. Date: Dec. 20, 2007

(54) Prevention of nuclear, solar, and other radiation-induced tissue damage

(75) Inventors: AbulKalam M. Shamsuddin, Lutherville, MD (US); Ivana Vucenik, Ellicott City, MD (US)

Correspondence Address: BINGHAM MCCUTCHEN LLP, 2020 K Street, N.W., Intellectual Property Department, WASHINGTON, DC, 20006, US

(73) Assignee: IP-6 Research Inc.

(21) Appl. No.: 11/453,843

(22) Filed: Jun. 16, 2006

(51) Int. Cl. A61K 31/66

(52) U.S. Cl. 514/102

(57) ABSTRACT

Inositol hexaphosphate (IP-6) is a polyphosphorylated carbohydrate with potent antioxidant activity to prevent active oxygen species-mediated mutagenesis, cell injury and carcinogenesis. IP-6 also activates DNA repair mechanisms. Sublethal radiation causes DNA damage through the formation of free radicals, reactive oxygen species, and pyrimidine crosslinks leading to cellular proliferation, cell cycle arrest and apoptosis. In the skin it results in the induction of skin cancer, premature skin aging, immuno-suppression, inflammation, and cell death. Likewise sublethal exposure to ionizing radiation as in nuclear blasts (war-time, accidental, terrorist-induced etc), cosmic radiation, etc. also causes the same spectrum of damage to the cells and the organisms with acute symptoms and eventual high risk of many cancers. IP-6 and/or inositol and their pharmaceutically acceptable salts and derivatives, including pyrophosphates and citrate derivatives, significantly counteract the harmful effects of radiation, affecting cell cycle progression in a protective manner (more cells in the protective GI phase) as well as decreasing apoptosis and caspase-3 activation. Various salts of IP-6 are used with comparable efficacy and the combination of IP-6+inositol affords the best protection against radiation-induced cell injury. Thus IP-6 and inositol are effective agents for protection against nuclear, solar and other radiation injuries.

### FIELD OF THE INVENTION

The invention relates to the field of protecting mammalian cells and tissues from the adverse effects of anticipated, planned or inadvertent exposure to radiation, particularly ionizing radiation. In particular, the present invention relates to the use of inositol hexaphosphate (IP-6) and its derivatives, including pyrophosphate and citrate derivatives, with or without inositol, administered to a mammalian subject or mammalian cells prior to, during, or after exposure to radiation for the prevention or treatment of damage to such mammals and their tissues and cells from exposure to solar, nuclear, cosmic, and other forms of electromagnetic or particulate radiation; including radiation exposure such as occurs during anticancer radiotherapy.

### BACKGROUND OF THE INVENTION

Radiation is energy distributed across the electromagnetic spectrum, interacting with matter in a way that may be described by reference to waves (having long wavelength and low frequency) and/or to particles (having short wavelength and high frequency). Approximately 80% of all radiation encountered normally by mammals is from naturally-occurring sources. Radiation, particularly ionizing radiation, has an adverse effect on cells and tissues, primarily through cytotoxic effects. In humans, exposure to ionizing radiation occurs primarily through therapeutic techniques (such as anticancer radiotherapy), through occupational and/or environmental exposure to human-derived radiation sources, or through occupational exposure to naturally-occurring radiation sources as in the case of aircraft flight personnel.

Table 1 characterizes radiation forms according to their frequency and selected biological effects. Properties of radiation forms closer to the low-frequency end of the spectrum are better described as wavelike. Radiation forms closer to the high-frequency end of the spectrum have the most energy and tend to interact with matter as particles. The hazardous effects of radiation exposure are typically associated with the particulate characteristics of the radiation type.

TABLE 1
Ionizing and Non-Ionizing Electromagnetic Radiation

| Radiation | Frequency (Hz) | Selected Biological Effects |
|---|---|---|
| Electrical power | 1–50 | Possible increased incidence |
| | | of cancer |
| Radio waves and radar | 106–1,011 | Thermal effects, cataracts |
| Microwaves | 109–1,010 | Lens opacities |
| Infrared | 1,011–1,014 | Cataracts |
| Visible light | 1,015 | Retinal burns (lasers) |
| Ultraviolet light | 1,015–1,018 | Skin burns, skin cancer |
| X-rays and gamma rays | 1,018–1,020 | Acute and delayed injury, |
| | | cancer |
| Cosmic radiation | 1,027 | Possible cataract, brain |
| | | damage, cancer |

Ionizing radiation is characterized as having short wavelength and high frequency; typical ionizing radiation forms include ultraviolet light, X-rays, gamma rays and cosmic radiation. Forms of ionizing radiation also include radioactive emissions of particles such as alpha particles or neutrons. In accord with its particulate nature, ionizing radiation causes vibration and rotation of atoms in biological molecules resulting in the ejection of electrons and the creation of free radical chemical species. Free radicals are chemical species that have a single unpaired electron in an outer orbit. This unstable configuration favors the release of energy through interactions with neighboring molecules, both inorganic and organic. In biomolecules, this release results in physical alteration of the subject biomolecules through the creation of aberrant chemical bonds. Thus, ionizing radiation may be said to exert a direct effect on biomolecules.

Free radical species may also be created through chemical, enzymatic, and catalytic means by way of an intermediate reactive substance, but ionizing radiation can create free radicals directly, for example by directly hydrolyzing water into hydroxyl (OH.) and hydrogen (H.) free radicals. For example, when tissues are exposed to gamma radiation, much of the energy deposited in the cells is absorbed by water and results in scission of the oxygen-hydrogen covalent bonds in water, leaving a single electron on hydrogen and another on oxygen, thus creating the two radicals. The hydroxyl radical (OH.) is the most reactive radical known in chemistry.

Free radical species react with biomolecules, such as the purine or pyrimidine bases of nucleic acids, proteins, lipids, and other biological macromolecules to produce damage to cells and tissues, and they can set off intra- and extra-cellular chain reactions, particularly in the critically ill patient. For example, reactive free-radical oxygen species initiate the activation of transcription factors through signal transduction from the cell surfaces, resulting in inflammation and tumor promotion.

It is generally accepted that DNA is the crucial target for the cytotoxic effects of ionizing radiation. Ionizing radiation is capable of damaging or altering DNA directly, causing double-stranded (ds) breaks and the formation of crosslinked pyrimidine bases, such as thymidine dimers, as particularly important by-products. Carbon-centered radicals formed directly by ionizing radiation on the deoxyribose moiety of DNA are thought to be the precursors of strand breaks. Cells undergoing extensive irreparable DNA damage generally enter into apoptosis (programmed cell death), and surviving cells bear the hallmarks of radiation damage in the form of mutations, chromosomal abnormalities and genetic instability.

Rapidly dividing cells, such as the blood forming cells (hematopoietic) in bone marrow, germ cells in testes and ovary, mucosal lining cells of the gastrointestinal tract, airway, etc. are most susceptible to injury from ionizing radiation. Cells in the G2 and mitotic phases of the cell cycle are the most likely to be damaged. In fact, it has been suggested that much of what is considered critical illness may involve oxygen radical (“oxyradical”) pathophysiology. Oxyradical injury has been implicated in the pathogenesis of pulmonary oxygen toxicity, adult respiratory distress syndrome (ARDS), bronchopulmonary dysplasia, sepsis syndrome, and a variety of ischemia-reperfusion syndromes, including myocardial infarction, stroke, cardiopulmonary bypass, organ transplantation, necrotizing enterocolitis, acute renal tubular necrosis, and other diseases.

Radiation exposure from any source can be classified as acute (a single large exposure) or chronic (a series of small low-level, or continuous low-level exposures spread over time). Table 2 sets forth the radiation doses from selected sources. Radiation dosage is generally reported in millirem.

TABLE 2

| Source | Dose In Millirem |
|---|---|
| Television | <1/yr |
| Gamma Rays, Jet Cross Country | 1 |
| Mountain Vacation - 2 week | 3 |
| Atomic Test Fallout | 5 |
| U.S. Water, Food & Air (Average) | 30/yr |
| Wood | 50/yr |
| Concrete | 50/yr |
| Brick | 75/yr |
| Chest X-Ray | 100 |
| Cosmic Radiation (Sea Level) | 40/yr (add 1 millirem/100 ft elev.) |
| Natural Background San Francisco | 120/yr |
| Natural Background Denver | 50/yr |
| Atomic Energy Commission | 5,000/yr |
| Limit For Workers | |
| Complete Dental X-Ray | 5,000 |
| Natural Background at Pocos de | 7,000/yr |
| Caldras, Brazil | |
| Whole Body Diagnostic X-Ray | 100,000 |
| Cancer Therapy | 500,000 (localized) |
| Radiation Sickness-Nagasaki | 125,000 (single doses) |
| LD.sub.50 Nagasaki & Hiroshima | 400,000–500,000 (single dose) |

Radiation sickness generally results from an acute exposure of a sufficient dose, and presents with a characteristic set of symptoms that appear in an orderly fashion, including hair loss, weakness, vomiting, diarrhea, skin burns and bleeding from the gastrointestinal tract and mucous membranes. Genetic defects, sterility and cancers (particularly bone marrow cancer) often develop over time. A sufficiently large acute dose of ionizing radiation, for example 500,000 to over 1 million millirem (equivalent to 5-10 Gy), may kill a subject immediately. Doses in the hundreds of thousands of millirems may kill within 7 to 21 days from a condition called “acute radiation poisoning.” An acute total body exposure of 125,000 millirem may cause radiation sickness. Localized doses such as are used in radiotherapy may not cause radiation sickness, but may result in the damage or death of exposed normal cells.

For example, an acute total body radiation dose of 100,000-125,000 millirem (equivalent to 1 Gy) received in less than one week would result in observable physiologic effects such as skin burns or rashes, mucosal and GI bleeding, nausea, diarrhea and/or excessive fatigue. Longer term cytotoxic and genetic effects such as hematopoietic and immunocompetent cell destruction, hair loss (alopecia), gastrointestinal, and oral mucosal sloughing, venoocclusive disease of the liver and chronic vascular hyperplasia of cerebral vessels, cataracts, pneumonitis, skin changes, and an increased incidence of cancer may also manifest over time. Acute doses of less than 10,000 millirem (equivalent to 0.1 Gy) typically will not result in immediately observable biologic or physiologic effects, although long term cytotoxic or genetic effects may occur.

Chronic exposure is usually associated with delayed medical problems such as cancer and premature aging. Chronic radiation exposure is a low level (i.e., 100-5,000 millirem) incremental or continuous radiation dose received over time. Examples of chronic doses include a whole body dose of about 5,000 millirem per year, which is the dose typically received by an adult human working at a nuclear power plant. By contrast, the Atomic Energy Commission recommends that members of the general public should receive no more than 100 millirem per year. Chronic doses may cause long-term cytotoxic and genetic effects, for example manifesting as an increased risk of a radiation-induced cancer developing later in life.

Chronic doses of greater than 5,000 millirem per year (0.05 Gy per year) may result in long-term cytotoxic or genetic effects similar to those described for persons receiving acute doses. Some adverse cytotoxic or genetic effects may also occur at chronic doses of significantly less than 5,000 millirem per year. For radiation protection purposes, it is assumed that any dose above zero can increase the risk of radiation-induced cancer (i.e., that there is no threshold). Epidemiologic studies have found that the estimated lifetime risk of dying from cancer is greater by about 0.04% per rem of radiation dose to the whole body.

A major source of (acute) exposure to ionizing radiation is the administration of human-derived therapeutic radiation in the treatment of cancer or other proliferative disorders. Subjects exposed to therapeutic doses of ionizing radiation typically receive between 0.1 and 2 Gy per treatment, and can receive as high as 5 Gy per treatment. Depending on the course of treatment prescribed by the treating physician, multiple doses may be received by a subject over the course of several weeks to several months.

Exposure to ionizing radiation from human-derived sources can also occur in the occupational setting. Occupational doses of ionizing radiation may be received by persons whose job involves exposure (or potential exposure) to radiation, for example in the nuclear power and nuclear weapons industries. Occupational exposure may also occur in rescue and emergency personnel called in to deal with catastrophic events involving a nuclear reactor or radioactive material. Other sources of occupational exposure may be from machine parts, plastics, solvents left over from the manufacture of radioactive medical products, smoke alarms, emergency signs, and other consumer goods. Occupational exposure may also occur in military or civilian persons who serve on nuclear powered vessels, particularly those who tend the nuclear reactors, and those operating in areas contaminated by military uses of radioactive materials, including nuclear weapons fallout.

Mammals, including humans and other animals (such as livestock), may also be exposed to ionizing radiation of human derivation from the environment. The primary source of exposure to significant amounts of such environmental radiation is from nuclear power plant accidents, such as those at Three Mile Island, Chemobyl and Tokaimura. Environmental exposure to ionizing radiation may also result from nuclear weapons detonations (either experimental or during wartime), discharges of actinides from nuclear waste storage and processing and reprocessing of nuclear fuel, and from naturally occurring radioactive materials such as radon gas or uranium. There is also increasing concern that the use of ordnance containing depleted uranium results in low-level radioactive contamination of combat areas.

As noted above, for most mammals the bulk of their lifetime radiation exposure derives from naturally-occurring sources. Such sources include radioactive chemical elements dispersed throughout nature, such as the small amount of uranium that occurs naturally in granite. Small amounts of radioactive elements are found pervasively in the atmosphere, ground, and water, to lesser and greater degrees depending upon location. Other significant naturally-occurring sources derive from outer space: the sun and the cosmos. Ultraviolet radiation emitted by the sun may be particularly hazardous as relatively strong doses may be acquired accidentally throughout much of the world. Cosmic radiation, x-ray, and gamma radiation exposure is of particular risk to those mammals living or working at high altitudes. Commercial and military flight personnel, including astronauts, are particularly susceptible to such radiation owing to the relatively long periods they spend at high-altitudes.

While anti-radiation suits or other protective gear may be effective at reducing radiation exposure, such gear is expensive, unwieldy, and generally not available to public. Moreover, radioprotective gear will not protect normal tissue adjacent a tumor from stray radiation exposure during radiotherapy. What is needed, therefore, is a practical way to protect subjects who are scheduled to incur, or are at risk for incurring, exposure to ionizing radiation. In the context of therapeutic irradiation, it is desirable to enhance protection of normal cells while causing tumor cells to remain vulnerable to the detrimental effects of the radiation. Furthermore, it is desirable to provide systemic protection from anticipated or inadvertent total body irradiation, such as may occur with occupational or environmental exposures, or with certain therapeutic techniques.

Pharmaceutical radioprotectants offer a cost-efficient, effective and easily available alternative to radioprotective gear. However, previous attempts at radioprotection of normal cells with pharmaceutical compositions have not been entirely successful. For example, cytokines directed at mobilizing the peripheral blood progenitor cells confer a myeloprotective effect when given prior to radiation (Neta et al., Semin. Radiat. Oncol. 6:306-320, 1996), but do not confer systemic protection. Other chemical radioprotectors administered alone or in combination with biologic response modifiers have shown minor protective effects in mice, but application of these compounds to large mammals was less successful, and it was questioned whether chemical radioprotection was of any value (Maisin, J. R., Bacq and Alexander Award Lecture. “Chemical radioprotection: past, present, and future prospects”, Int. J. Radiat. Biol. 73:443-50, 1998).

In today's heightened nuclear threat from terrorists and/or rogue nations as well as accidents in nuclear power-plant reactors, there is an increased need to have safe and effective means to protect the nuclear-reactor workers and the population at large from the health hazards of ionizing radiation exposures. The United States Department of Energy (DOE) reports that “an unfilled dream of civil and military officials concerned with this issue is to have a globally effective pharmacologic, i.e. the magic radioprotective pill. This pill could be taken orally without any undue side effects prior to or after a suspected nuclear/radiological event in order to provide the individual full bodily protection against early arising acute injury and late arising pathologies. (United States Department of Energy Report of Jul. 13, 2005 on Inositol and Other Radioprotective Agents Workshop).” Thus, a “radioprotective pill” is of urgent and vital national security interest.

The DOE report further states: “ . . . . Currently a full range of R&D strategies are being employed in the hunt for new safe and effective radioprotectants including: a) large scale screening of newly identified chemical classes or natural products; b) reformulating or restructuring older protectants with proven efficacies to reduce unwanted toxicities; c) using nutraceuticals that are only moderately protective but that are essentially non-toxic and exceedingly well tolerated; d) using low dose combinations of potentially toxic (at high drug doses) but efficacious agents that cytoprotect through different routes in hopes of fostering radioprotective synergy; and e) accepting lower drug efficacy in lieu of non-toxicity, banking on the protection afforded by the drug can be leveraged by post-exposure therapies . . . .” However, as regards IP-6 and its derivatives, including pyrophosphates, and/or inositol, the subject matter of this application, those skilled in the art conclude: “Inositol hexaphosphate, IP-6, and its analogs are entering testing as drugs. One of the challenges is to cover phosphates with protecting groups, to facilitate passage of the molecule into the cell. (DOE report of Jul. 13, 2005).” Implied is the notion that IP-6 and its derivatives, including pyrophosphates, and/or inositol may currently be ineffective as radioprotectants.

Prior art exploration of radio-modifiers, such as radio-protectors and radio-sensitizers, has focused on hypoxic cell sensitizers such as metranidazole and misonidazole. Radio-protectors have received much less attention than radio-sensitizers at the clinical level. The nuclear era spawned considerable effort in the development of radio-protectors with more than 4,000 compounds being synthesized and tested at the Walter Reed Army Institute of Research in the United States of America in the 1960's. With the exception of a compound known as WR2727, none of those compounds has proven useful in either the military or industrial contexts or for cancer radiotherapy.

### SUMMARY OF THE INVENTION

The present invention provides compositions and methods for protecting the normal cells and tissues from the cytotoxic and genetic effects of exposure to radiation, particularly ionizing radiation, in subjects who have incurred or are at risk of incurring exposure to ionizing radiation. The exposure to ionizing radiation may occur in controlled doses during the treatment of cancer and other proliferative disorders, or may occur in uncontrolled doses beyond the norm accepted for the population at large during high risk activities or environmental exposures.

Thus in one aspect, inositol/IP-6 compounds and pharmaceutically acceptable salts and derivatives, including pyrophosphate and citrate derivatives, and pharmaceutical compositions comprising the same are provided.

The present invention provides a method for preventing or treating acute short-term adverse health effects of ionizing radiation exposure in a mammal, comprising: administering to the mammal an effective amount of a pharmaceutical composition comprising IP-6, its pharmaceutically acceptable salts, or its pharmaceutically acceptable derivatives, in any combination; and preventing or treating acute short-term adverse health effects of ionizing radiation exposure in a mammal. In a preferred embodiment, the pharmaceutical composition further comprises inositol. In another embodiment, the pharmaceutical composition further comprises at least one pharmaceutically acceptable excipient or carrier, and it may be provided in the form of a liquid, lotion, cream, gel, ointment, powder, tablet, chewable tablet, suppository, or capsule. The pharmaceutical composition may be an enteral or a parenteral formulation.

In another embodiment, the pharmaceutical composition contains IP-6, its pharmaceutically acceptable salts, or its pharmaceutically acceptable derivatives, in any combination, in an amount from about 0.1% to about 100% by weight, or in from about 0.1% to about 50% by weight wherein it further comprises inositol, its pharmaceutically acceptable salts, or its pharmaceutically acceptable derivatives, in any combination, in an amount from about 0.1% to about 50% by weight. In a preferred embodiment, the pharmaceutical composition contains inositol and IP-6, their pharmaceutically acceptable salts, or their pharmaceutically acceptable derivatives, in any combination, in a ratio of about 30:1 to about 1:30 or in a ratio of about 5:1 to about 1:5.

In another embodiment, the pharmaceutical composition contains IP-6, its pharmaceutically acceptable salts, or its pharmaceutically acceptable derivatives, in any combination, in an amount from about 0.01% to about 20% by weight. Preferably, the pharmaceutical composition contains IP-6, its pharmaceutically acceptable salts, or its pharmaceutically acceptable derivatives, in any combination, in an amount from about 0.01% to about 20% by weight, and further comprises inositol, its pharmaceutically acceptable salts, or its pharmaceutically acceptable derivatives, in any combination, in an amount from about 0.01% to about 20% by weight. Even more preferably, the composition contains from about 0.1% to about 10% by weight of IP-6 and further comprises inositol, its pharmaceutically acceptable salts, or its pharmaceutically acceptable derivatives, in any combination, in an amount from about 0.1% to about 10% by weight. In another embodiment, the pharmaceutical composition contains IP-6, its pharmaceutically acceptable salts, or its pharmaceutically acceptable derivatives, in any combination, in an amount from about 0.5% to about 5% by weight, and further comprises inositol, its pharmaceutically acceptable salts, or its pharmaceutically acceptable derivatives, in any combination, in an amount from about 0.5% to about 5% by weight. Preferably, the pharmaceutical composition contains inositol and IP-6, their pharmaceutically acceptable salts, or their pharmaceutically acceptable derivatives, in any combination, in a ratio of about 30:1 to about 1:30 or in a ratio of about 5:1 to about 1:5, and the inositol and IP-6 may be present in the forms of pharmaceutically acceptable salts, isomers, esters, derivatives, or any combination thereof.

The pharmaceutical composition may also contain an antioxidant, a biocide, a chemotherapeutic, a nutritional supplement or nutraceutical, an analgesic, a sunblock, a moisturizer, or any combination thereof. The pharmaceutical composition may be administered orally as a powder, tablet, or capsule and topically as a lotion, cream, ointment or gel.

In one embodiment, the pharmaceutical composition is administered for at least one day prior to ionizing radiation exposure. Preferably, the administration is performed at least twice daily or at least three times daily, and the pharmaceutical composition is administered in at least one dose containing a total of about 1 gram to about 10 grams of inositol, IP-6, their pharmaceutically acceptable salts, or their pharmaceutically acceptable derivatives, in any combination. In another embodiment, the pharmaceutical composition is administered in at least one dose containing about 2 gram to about 5 grams of inositol, IP-6, their pharmaceutically acceptable salts, or their pharmaceutically acceptable derivatives, in any combination.

The present invention provides that the ionizing radiation exposure comprises ultraviolet light, x-rays, gamma rays, cosmic rays, particle beams, or any combination thereof, wherein the ionizing radiation derives from one or more natural sources, including: the sun, outer space, or radioactive elements present in the atmosphere, ground, mineral deposits, mined ore, groundwater, bodies of water, or stone. The ionizing radiation may also be derived from one or more human-derived sources, such as: ultraviolet lights, therapeutic radiation sources, nuclear power plants, nuclear fuel, nuclear weapons, nuclear fallout, and radioactive consumer devices.

The adverse health effects prevented or reduced by compositions of the present invention include, among others: skin burns, rashes, mucosal degradation or bleeding, gastro-intestinal degradation or bleeding, diarrhea, anemia, or excessive fatigue.

The present invention also provides a method for safely increasing the dosage of therapeutic ionizing radiation provided to a mammal in need of ionizing radiation therapy, comprising: administering to a mammal prior to exposure to therapeutic radiation ionizing radiation a pharmaceutical composition comprising a composition as provided above and exposing the mammal to a dosage of therapeutic radiation ionizing radiation greater than a maximum safe dosage for said mammal in the absence of said pharmaceutical composition.

The present invention also provides a method for protecting a worker from short-term adverse health effects of workplace ionizing radiation exposure, comprising: administering to a worker prior to exposure to workplace ionizing radiation a pharmaceutical composition comprising a composition as provided above; and protecting the worker from adverse health effects of workplace ionizing radiation exposure.

The present invention also provides a method for protecting military personnel from short-term adverse health effects of human-derived ionizing radiation exposure, comprising: administering to military personnel prior to exposure to military ionizing radiation a pharmaceutical composition comprising a composition as provided above; and protecting the military personnel from adverse health effects of human-derived ionizing radiation exposure.

The present invention also provides a kit used for protecting military personnel from short-term adverse health effects of human-derived ionizing radiation exposure, comprising: a container, containing a plurality of pharmaceutical compositions comprising IP-6, its pharmaceutically acceptable salts, or its pharmaceutically acceptable derivatives, in any combination, said plurality of pharmaceutical compositions comprising a topical preparation and an oral preparation effective to protect military personnel from short-term adverse health effects of human-derived ionizing radiation exposure. Preferably, the plurality of pharmaceutical compositions are provided in unit doses and the kit contains sufficient unit doses for at least one day's usage. More preferably, the plurality of pharmaceutical compositions further comprises inositol, its pharmaceutically acceptable salts, or its pharmaceutically acceptable derivatives, in any combination. In another embodiment, the plurality of pharmaceutical compositions are administered in at least one dose each containing a total of about 1 gram to about 10 grams of inositol, IP-6, their pharmaceutically acceptable salts, or their pharmaceutically acceptable derivatives, in any combination.

The present invention also provides a topical preparation for preventing acute short-term adverse health effects of ionizing radiation exposure in a mammal, comprising: an effective amount of a composition comprising IP-6, its pharmacologically acceptable salts, or its pharmacologically acceptable derivatives, in any combination, and at least one pharmacologically acceptable carrier, effective to prevent acute short-term adverse health effects of ionizing radiation exposure in a mammal. Preferably, the composition is a lotion, cream, or gel, and the acute short-term adverse health effects of ionizing radiation exposure comprise sunburn. Preferably, the composition is applied to the skin of the mammal at a time sufficiently prior to the ionizing radiation exposure to allow the IP-6, its pharmacologically acceptable salts, or its pharmacologically acceptable derivatives, in any combination, to be absorbed by cells of the skin. Even more preferably, the pharmaceutical composition is applied to the skin of the mammal three to twelve hours prior to the ionizing radiation exposure.

In another embodiment, the topical preparation described above also includes an antioxidant, a biocide, a nutritional supplement or nutraceutical, an analgesic, a sunblock, a moisturizer, or any combination thereof, and even further contains inositol, its pharmaceutically acceptable salts, or its pharmaceutically acceptable derivatives, in any combination. In one embodiment, the composition contains IP-6, its pharmaceutically acceptable salts, or its pharmaceutically acceptable derivatives, in any combination, in an amount from about 0.1% to about 50% by weight, and further comprises inositol, its pharmaceutically acceptable salts, or its pharmaceutically acceptable derivatives, in any combination, in an amount from about 0.1% to about 50% by weight. In another embodiment, the composition contains inositol and IP-6, their pharmaceutically acceptable salts, or their pharmaceutically acceptable derivatives, in any combination, in a ratio of about 30:1 to about 1:30 or in a ratio of about 5:1 to about 1:5.

In another aspect, a method of treating a subject for cancer or other proliferative disorders is provided, comprising administering to the subject an effective amount of at least one radioprotectant inositol/IP-6 compound prior to administering an effective amount of ionizing radiation, wherein the inositol/IP-6 compound induces a temporary radioresistant phenotype in the subject's normal tissue.

In yet another embodiment, the invention provides a method for purging bone marrow of neoplastic cells (such as leukemic cells) or tumor cells which have metastasized into the bone marrow, comprising harvesting bone marrow cells from an individual afflicted with a proliferative disorder, treating the harvested bone marrow cells with an effective amount of at least one inositol/IP-6 compound, and subjecting the treated bone marrow cells with to an effective amount of ionizing radiation. The harvested cells are then returned to the body of the afflicted individual.

In yet a further aspect, the invention provides a method for treating individuals who have incurred or are at risk for incurring remediable radiation damage from exposure to ionizing radiation. In one embodiment, an effective amount of at least one inositol/IP-6 compound is administered to the subject before the subject incurs remediable radiation damage from exposure to ionizing radiation. In another embodiment, an effective amount of at least one inositol/IP-6 compound is administered to the subject after the subject incurs remediable radiation damage from exposure to ionizing radiation.

### BRIEF DESCRIPTION OF THE FIGURES

FIG. 1. Effect of IP-6 and UVB radiation on HaCaT cell viability and proliferation. HaCaT cells were exposed to different UVB intensities, and immediately treated with various concentrations of Na-IP-6. Cell viability and proliferation was determined 24 h later using the MTT assay. Each data point represents the mean ± standard deviation. There was a significant increase in relative cell viability with increasing IP-6 concentrations, p<0.001. The concentration of inositol in the media is 38.8 μM.

FIG. 2. Effect of IP-6 and UVB radiation on attachment of HaCaT cells. HaCaT cells were exposed to different UVB intensities, and treated immediately with different concentrations of Na-IP-6. Cell attachment was determined 18 h following UVB radiation. Each data point represents the mean ± standard deviation. At 30 mJ/cm² UVB intensity, IP-6 concentrations of 0.5, 1.0 and 2.0 mM caused a significant increase in cell attachment, p<0.01. The concentration of inositol in the media is 38.8 μM.

FIG. 3. Effect of IP-6 and UVB radiation on cell cycle distribution of HaCaT cells. For annexin V and PI staining cells were either not exposed or exposed to 30 mJ/cm² UVB radiation, then either treated with 1.0 mM Na-IP-6 or not treated with IP-6. Eighteen hours later, cells were harvested and double stained with annexin V and PI and analyzed by flow cytometry. The number of cells that are present in apoptosis, necrosis or are viable are represented as percentages; p<0.001, as compared to the group exposed to UVB but not treated with IP-6. The concentration of inositol in the media is 38.8 μM.

FIG. 4. Effect of IP-6 and UVB radiation on caspase-3 activation of HaCaT cells. To assess activated caspase-3 activity, cells were either not exposed or exposed to 30 mJ/cm² UVB radiation, and then either treated with 1.0 mM Na-IP-6 or not treated with IP-6. Eighteen hours later, cells were harvested and fluorometric CaspACE assays were performed. Activated caspase-3 activity is represented as relative fluorescence units. **Significant at p<0.01, as compared to the group exposed to UVB but not treated with IP-6. The concentration of inositol in the media is 38.8 μM.

FIG. 5: Following UVB exposure, as signs of cellular injury and death, the control untreated cells show less attachment to the plate as opposed to those treated with Na-IP-6, Inositol (Ins) and IP-6+Inositol. Cells treated with 1:1 molar ratio of IP-6 and Inositol showed the best attachment.

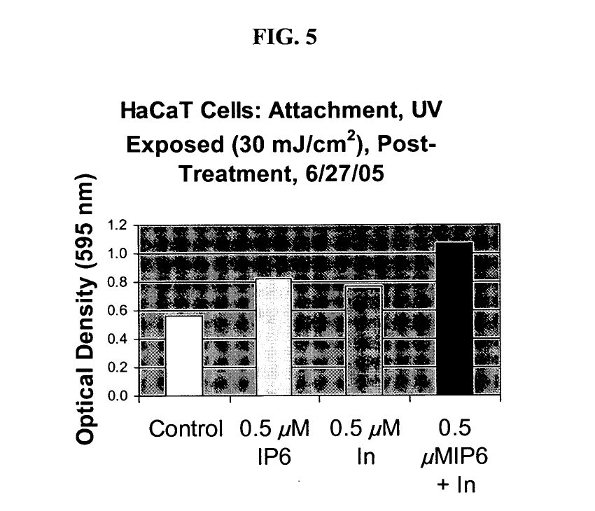

### DETAILED DESCRIPTION OF THE INVENTION

Inositol (hexahydroxycyclohexane) and its phosphates are well known, nontoxic, naturally occurring compounds. Inositol is a 6-carbon sugar that is present in both animal and plant cells, either as a part of larger molecules, e.g. phospholipids, or in phosphorylated form, e.g. the various inositol phosphates, polyphosphates, and pyrophosphates. The inositols can be commercially prepared by a number of methods, e.g. see Postemak U.S. Pat. No. 1,313,014; Goedecke U.S. Pat. No. 1,715,031; Wagner, U.S. Pat. No. 1,716,286; Goedecke U.S. Pat. No. 1,721,214; U.S. Pat. No. 2,112,553; Elkin, U.S. Pat. No. 2,414,365; etc.

The inositol/IP-6 compounds and compositions of the invention protect normal cells and tissues from the effects of acute and chronic exposure to radiation, particularly ionizing radiation. The term “inositol/IP-6” as used herein refers to the inositol-based compounds of the present invention, including inositol, IP-6, pharmacologically acceptable derivatives of IP-6, including pyrophosphate and citrate derivatives of IP-6, for example IP-7, IP-8, IP-12, and IP-6 hexacitrate, etc., any prodrugs of the same, and pharmacologically acceptable salts thereof, in any efficacious combination.

The term “subject” includes human beings and non-human animals and, as used herein, refers to an organism which is scheduled to incur, is at risk of incurring, or has incurred, exposure to ionizing radiation.

As used herein, “ionizing radiation” is radiation of sufficient energy that, when absorbed by cells and tissues, induces formation of free radical species and DNA damage. This type of radiation includes ultraviolet radiation, X-rays, gamma rays, and particle bombardment (e.g., neutron beam, electron beam, protons, mesons and others), and is used for medical testing and treatment, scientific purposes, industrial testing, manufacturing and sterilization, weapons and weapons development, and many other uses. Radiation is typically measured in units of absorbed dose, such as the rad or gray (Gy), or in units of dose equivalence, such as the rem or sievert (Sv).

The Sv is the Gy dosage multiplied by a factor that includes tissue damage done. For example, penetrating ionizing radiation (e.g., gamma and beta radiation) has a factor of about 1, so 1 Sv equal to about 1 Gy. Alpha rays have a factor of 20, so 1 Gy of alpha radiation is equal to 20 Sv.

By “effective amount of ionizing radiation” is meant an amount of ionizing radiation effective in killing, or in reducing the proliferation of, abnormally proliferating cells in a subject. As used with respect to bone marrow purging, “effective amount of ionizing radiation” means an amount of ionizing radiation effective in killing, or in reducing the proliferation, of malignant cells in a bone marrow sample removed from a subject.

By “acute exposure to ionizing radiation” or “acute dose of ionizing radiation” is meant a dose of ionizing radiation absorbed by a subject in less than 24 hours. The acute dose may be localized, as in radiotherapy techniques, or may be absorbed by the subject's entire body. Acute doses are typically above 10,000 millirem (0.1 Gy), but may be lower.

The term “acute radiation-induced skin damage” refers to the damage to the epidermal layer of the skin caused by either a single large dosage or repeated smaller dosages of ionizing radiation, which damage can manifest itself within at least about 3-5 weeks following exposure and often much earlier. Acute radiation-induced skin damage may occur shortly after exposure to ionizing radiation, as in the case of sunburn. Acute radiation-induced skin damage is sometimes referred to as early radiation induced skin damage and includes, by way of example, erythema, dry desquamation, moist desquamation, epilation and ulceration. Acute radiation-induced skin damage can be particularly severe in skin folds and areas of high friction, e.g., groin, buttocks, the folds of the breast, neck, etc. and the like.

The term “late radiation-induced skin damage” refers to skin damage arising 6 or more months after exposure to the radiation, which damage includes, by way of example, atrophy, fibrosis, thinning, telangiectasia, altered pigmentation, ulceration, necrosis and carcinogenesis.

By “chronic exposure to ionizing radiation” or “chronic dose of ionizing radiation” is meant a dose of ionizing radiation absorbed by a subject over a period greater than 24 hours. The dose may be intermittent or continuous, and may be localized or absorbed by the subject's entire body. Chronic doses are typically less than 10,000 millirem (0.1 Gy), but may be higher.

By “at risk of incurring exposure to ionizing radiation” is meant that a subject may advertently (such as by scheduled radiotherapy sessions) or inadvertently be exposed to ionizing radiation in the future. Inadvertent exposure includes accidental or unplanned environmental or occupational exposure.

By “effective amount of the inositol/IP-6 compound” is meant an amount of compound effective to reduce or eliminate the toxicity associated with radiation in normal cells of the subject. A second benefit may also be found in some cases when an “effective amount of the inositol/IP-6 compound” is used; inositol/IP-6 compounds in effective amounts in such cases have antineoplastic, apoptosis-inducing, and cell differentiating effects. As used with respect to bone marrow purging, “effective amount of the inositol/IP-6 compound” means an amount of inositol/IP-6 compound effective to reduce or eliminate the toxicity associated with radiation in bone marrow removed from a subject, and also to impart a direct cytotoxic or antineoplastic effect to malignant cells in the bone marrow removed from the subject.

As used herein, a “prodrug” is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The precise radioprotectant mechanism of action of the inositol/IP-6 compositions on normal cells is unknown. However, based on experimental models, and without wishing to be bound by any theory, these compounds may affect several elements in normal cells which induce a reversible quiescent cell-cycling state in which transit through mitosis, and many of the changes necessary for such passage, are down regulated, inactivated or absent. Conversely, in aberrant cells, particularly cancerous cells and damaged cells, these compounds may affect several elements that induce apoptosis of the aberrant or damaged cell, reactivate a stalled cell cycle or normalize an abnormal cell cycle, or induce differentiation of a transformed cell. According to other possible mechanisms of protection, radiation-induced reactive oxygen molecules, DNA damage, and activation of death-pathway induction may be rendered innocuous by pre-exposure to inositol/IP-6 compositions. Furthermore, inositol/IP-6 compositions provide a chemoprotective effect against “radiomimetic” drugs. Radiomimetic drugs are compounds that induce DNA damage and/or generation of oxygen radicals in the cell, analogous to ionizing radiation.

Although both mitotic phase inhibitors and ionizing radiation may cause cell cycle abnormalities, particularly cell cycle arrest, mitotic phase cell cycle inhibitors affect cells differently than ionizing radiation. For example, the mitotic phase cell cycle inhibitors do not cause cell death by DNA damage, and do not allow the cell to proceed past the G1 phase. Ionizing radiation damages DNA and typically causes cell cycle arrest in the G2 phase. Also, cells exposed to mitotic phase cell cycle inhibitors do not exhibit damage in the long term, but show only acute effects. By contrast, some effects from ionizing radiation may not be evident until at least two weeks after exposure, with damage to bone marrow appearing after 30 days, and neurologic damage manifesting up to six months after exposure. Effective amounts of the inositol/IP-6 compound of the present invention counteract these adverse effects.

Subjects may be exposed to ionizing radiation when undergoing therapeutic irradiation for the treatment of proliferative disorders. Such disorders included cancerous and non-cancer proliferative disorders. For example, the present compounds are believed effective in protecting normal cells during therapeutic irradiation of a broad range of tumor types, including but not limited to the following: breast, prostate, ovarian, lung, colorectal, brain (i.e., glioma) and renal tumors. The compounds are also effective against leukemic cells and in protecting non-cancerous cells from the adverse effects of radiation used to treat leukemia.

The compounds are also believed useful in protecting normal cells during therapeutic irradiation of abnormal tissues in non-cancer proliferative disorders, including but not limited to the following: hemangiomatosis in new born, secondary progressive multiple sclerosis, chronic progressive myelodegenerative disease, neurofibromatosis, ganglioneuromatosis, keloid formation, Paget's Disease of the bone, fibrocystic disease of the breast, Peronies and Dupuytren's fibrosis, restenosis and cirrhosis.

According to the invention, therapeutic ionizing radiation may be administered to a subject on any schedule and in any dose consistent with the prescribed course of treatment, as long as the inositol/IP-6 radioprotectant compound is administered prior to, during, and/or following the radiation exposure. The course of treatment differs from subject to subject, and those of ordinary skill in the art can readily determine the appropriate dose and schedule of therapeutic radiation in a given clinical situation.

Preferably, the inositol/IP-6 compound should be administered far enough in advance of the therapeutic radiation such that the compound is able to reach the normal cells of the subject in sufficient concentration to exert a radioprotective effect on the normal cells at the time of radiation exposure. The inositol/IP-6 compound may be administered as much as about 24 hours, preferably no more than about 18 hours, prior to administration of the radiation. In one embodiment, the inositol/IP-6 composition is administered at least about 6-12 hours before administration of the therapeutic radiation. In another embodiment, the inositol/IP-6 composition is administered once at about 2-3 hours before the radiation exposure. Most preferably, the inositol/IP-6 compound is administered once at about 18 hours and again at about 2-3 hours before the radiation exposure. One or more inositol/IP-6 compositions may be administered simultaneously, or different inositol/IP-6 compositions may be administered at different times during the treatment.

Where the therapeutic radiation is administered in serial fashion, it is preferable to intercalate administration of one or more inositol/IP-6 compositions within the schedule of radiation treatments. As above, different inositol/IP-6 compositions may be administered either simultaneously or at different times during the treatment. Preferably, an about 6 to 18 hour period separates administration of the final dose of inositol/IP-6 and each therapeutic radiation exposure. More preferably, the administration of the final dose of inositol/IP-6 and the therapeutic radiation is separated by about 2-3 hours. This strategy will yield significant reduction in radiation-induced side effects without adversely affecting the anticancer activity of the therapeutic radiation.

For example, therapeutic radiation at a dose of 0.1 Gy may be given daily for five consecutive days, with a two day rest, for a total period of 6-8 weeks. One or more inositol/IP-6 compositions may be administered to the subject 2-3 hours prior to each round of radiation, and more preferably once at about 12-24 hours and again at 2-3 hours prior to each round of radiation. It should be pointed out, however, that more aggressive treatment schedules, i.e., delivery of a higher dosage, is contemplated according to the present invention due to the protection of the normal cells afforded by the inositol/IP-6 compositions. Thus, the radioprotective effect of the inositol/IP-6 increases the therapeutic index of the therapeutic radiation, and may permit the physician to safely increase the dosage of therapeutic radiation above presently recommended levels without risking increased damage to the surrounding normal cells and tissues.

The inositol/IP-6 compositions of the invention are further useful in protecting normal bone marrow cells from radiologic treatments designed to destroy hematologic neoplastic cells or tumor cells which have metastasized into the bone marrow. Such cells include, for example, myeloid leukemia cells. The appearance of these cells in the bone marrow and elsewhere in the body is associated with various disease conditions, such as the French-American-British (FAB) subtypes of acute myelogenous leukemias (AML), chronic myeloid leukemia (CML), and acute lymphocytic leukemia (ALL). CML, in particular, is characterized by abnormal proliferation of immature granulocytes (e.g., neutrophils, eosinophils, and basophils) in the blood, bone marrow, spleen, liver, and other tissues and accumulation of granulocytic precursors in these tissues. The subject who presents with such symptoms will typically have more than 20,000 white blood cells per microliter of blood, and the count may exceed 400,000. Virtually all CML patients will develop “blast crisis”, the terminal stage of the disease during which immature blast cells rapidly proliferate, leading to death.

Other subjects suffer from metastatic tumors, and require treatment with total body irradiation (TBI). Because TBI will also kill the subject's hematopoietic cells, a portion of the subject's bone marrow is removed prior to irradiation for subsequent reimplantation. However, metastatic tumor cells are likely present in the bone marrow, and reimplantation often results in a relapse of the cancer within a short time.

Subjects presenting with neoplastic diseases of the bone marrow or metastatic tumors may be treated by removing a portion of the bone marrow (also called “harvesting”), purging the harvested bone marrow of malignant stem cells, and reimplanting the purged bone marrow. Preferably, the subject is simultaneously treated with radiation or some other anti-cancer therapy.

Thus, the invention provides a method of reducing the number of malignant cells in bone marrow, comprising the steps of removing a portion of the subject's bone marrow, administering an effective amount of at least one inositol/IP-6 compound and irradiating the treated bone marrow with a sufficient dose of ionizing radiation such that neoplastic or tumor cells in the bone marrow are killed. As used herein, “malignant cell” means any uncontrollably proliferating cell, such a tumor cell or neoplastic cell. The inositol/IP-6 compound protects the normal hematopoietic cells present in the bone marrow from the deleterious effects of the ionizing radiation. The inositol/IP-6 composition also exhibits a direct killing, antineoplastic, and/or differentiating effect on the malignant cells. The number of malignant cells in the bone marrow is significantly reduced or eliminated prior to reimplantation, thus minimizing the occurrence of a relapse.

Preferably, each dose of inositol/IP-6 is administered in a concentration from about 0.05 to about 100 millimolar. Dosages may be dependent upon the route of administration, with topical or enteral formulations generally having higher concentrations than parenteral formulations. Topical formulations may contain inositol/IP-6 in concentrations of about 10 to about 100 millimolar, more preferably, from about 10 to about 50 millimolar, and even more preferably from about 20 to about 40 millimolar. Particularly preferred concentrations are 20, 25, 30, 35, 40, 45, and 50 millimolar. Topical formulations may also contain inositol/IP-6 in amounts of about 0.5 to about 5% by weight, about 1 to about 4% by weight, or more preferably about 1.5 to about 3% by weight. In topical compositions containing inositol and IP-6, the ratio of inositol to IP-6 is preferably between about 1:1 to about 1:50, more preferably between about 1:10 to about 1:40, and even more preferably about 1:20 to about 1:30. Particularly preferred amounts are about 1:20, 1:25, 1:30, 1:35, and 1:40% by weight. Higher and lower amounts may also be used. In particular, oral formulations (tablet, capsule, powder, etc.) may also contain inositol/IP-6 at 50, 60, 70, 80, 90, 95, 99, and 100% by weight. In such cases, the total amount of inositol/IP-6 administered in a single dose will be about 1 to about 10 grams, preferably about 2 to about 5 grams, and even more preferably about 3 to about 4 grams. It should be noted that a single dose may comprise any number of individual capsules or tablets so long as the total amount of inositol/IP-6 is within the ranges specified. Preferably, tablets and capsules will contain about 1-2 grams of inositol/IP-6 each. Dosages may also be used at about 10, 15, 20, 25, 30, 35, 40, 45, and 50 mg/kg body weight of the subject, and range from about 10 to about 50, about 20 to about 40, and more preferably about 25 to about 35 mg/kg body weight.

For parenteral administration and in vivo usage, each dose of inositol/IP-6 is administered in a concentration from about 0.5 to about 50 millimolar. Parenteral and in vivo formulations may contain inositol/IP-6 in concentrations of about 1 to about 10 millimolar, more preferably, from about 2 to about 8 millimolar, and even more preferably from about 3 to about 5 millimolar. Particularly preferred concentrations are 0.5, 1, 1.5, 2, 2.5, 3, 3.5, 4, and 5 millimolar. Parenteral formulations may also contain inositol/IP-6 in amounts of about 0.05 to about 10% by weight, about 0.1 to about 10% by weight, or more preferably about 0.5 to about 5% by weight. Particularly preferred concentrations are 0.5, 1, 1.5, 2, 2.5, 3, 3.5, 4, and 5% by weight. In parenteral compositions containing inositol and IP-6, the ratio of inositol to IP-6 is preferably between about 5:1 to about 1:30, more preferably between about 2:1 to about 1:10, and even more preferably about 1:1 to about 1:5. Particularly preferred amounts are about 2:1, 1:1, 1:2, 1:3, and 1:4% by weight. Higher and lower amounts may also be used. In particular, amounts required to raise the in vivo (circulating or body) concentration to about 50 to about 300 micromolar, and preferably to about 100 to about 200 micromolar, may be used. In all embodiments, amounts may be adjusted to compensate for differences in amounts of active ingredients actually delivered to the treated cells or tissues.

It will be recognized by one of skill in the art that the optimal quantity and spacing of individual dosages of inositol/IP-6 compositions or pharmaceutically acceptable salts thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of inositol/IP-6 compositions or pharmaceutically acceptable salts thereof given per day for a defined number of days, can be ascertained by those skilled in the art using conventional course of treatment determination tests.

The inositol/IP-6 compositions may be added directly to harvested bone marrow or other cells in vitro but are preferably dissolved in water prior to addition. Pharmaceutical formulations of inositol/IP-6 such as are described in more detail below may also be used.

Preferably, the inositol/IP-6 composition is added to the harvested bone marrow or other cells or tissues about 20 hours prior to radiation exposure, preferably no more than about 24 hours prior to radiation exposure, and more preferably no more than 18 hours prior to exposure. In one embodiment, the inositol/IP-6 composition is administered to the harvested bone marrow or other cells at least about 6 hours before radiation exposure. In another embodiment, the inositol/IP-6 composition is administered at least about 2-3 hours before radiation exposure. In yet another embodiment, the inositol/IP-6 compound is administered at least about one half hour before radiation exposure. One or more inositol/IP-6 compositions may be administered simultaneously, or different inositol/IP-6 compositions may be administered at different times. Other dosage regimens may also be used.

If a subject is to be treated with ionizing radiation prior to reimplantation of purged bone marrow or other cells or tissues, the subject may be treated with one or more inositol/IP-6 compositions prior to, during, or after receiving the ionizing radiation dose, as described above.

A subject may also be exposed to ionizing radiation from occupation or environmental sources, as discussed in the background section. For purposes of the invention, the source of the ionizing radiation is not as important as the exposure type (i.e., acute or chronic) and dose level absorbed by the subject. It is understood that the following discussion encompasses ionizing radiation exposures from both occupational and environmental sources, and from both human-derived and naturally-occurring sources.

Subjects suffering from effects of acute or chronic exposure to ionizing radiation that are not immediately fatal are said to have remediable radiation damage. Such remediable radiation damage can be reduced or eliminated by the compounds and methods of the present invention.

An acute dose of ionizing radiation which may cause remediable radiation damage includes a localized or whole body dose, for example, between about 10,000 millirem (0.1 Gy) and about 1,000,000 millirem (10 Gy) in 24 hours or less, preferably between about 25,000 millirem (0.25 Gy) and about 200,000 (2 Gy) in 24 hours or less, and more preferably between about 100,000 millirem (1 Gy) and about 150,000 millirem (1.5 Gy) in 24 hours or less.

A chronic dose of ionizing radiation which may cause remediable radiation damage includes a whole body dose of about 100 millirem (0.001 Gy) to about 10,000 millirem (0.1 Gy), preferably a dose between about 1,000 millirem (0.01 Gy) and about 5,000 millirem (0.05 Gy) over a period greater than 24 hours, or a localized dose of 15,000 millirem (0.15 Gy) to 50,000 millirem (0.5 Gy) over a period greater than 24 hours.

The invention therefore provides a method for treating individuals who have incurred remediable radiation damage from acute or chronic exposure to ionizing radiation, comprising reducing or eliminating the cytotoxic effects of radiation exposure on normal cells and tissues by administering an effective amount of at least one inositol/IP-6 compound. The compound is preferably administered in as short a time as possible following radiation exposure, for example between 0-6 hours following exposure, and more preferably is followed by additional treatments every 2-6 hours, every 6-12 hours, or every day for a period of 1, 2, 3, 4, 5, 6, 7, 14, 21, or 28 days post-exposure.

Remediable radiation damage may take the form of cytotoxic and genotoxic (i.e., adverse genetic) effects in the subject. In another embodiment, there is therefore provided a method of reducing or eliminating the cytotoxic and genotoxic effects of radiation exposure on normal cells and tissues, comprising administering an effective amount of at least one inositol/IP-6 compound prior to, during, or following acute or chronic radiation exposure. The inositol/IP-6 compound may be administered, for example, about 24 hours prior to radiation exposure, preferably no more than about 18 hours prior to radiation exposure, and even more preferably no more than 6-12 hours prior to radiation exposure. The inositol/IP-6 compound may be administered during or immediately following radiation exposure, and preferably is further administered every 2-6 hours, every 6-12 hours, or every day for a period of 1, 2, 3, 4, 5, 6, 7, 14, 21, or 28 days post-exposure. In one embodiment, the inositol/IP-6 is administered at least about 6 hours or at least 2-3 hours before radiation exposure. More preferably, the inositol/IP-6 is administered at about 18 and again at about 2-3 hours before the radiation exposure, and even more preferably is administered again immediately following and every 2-3 post-exposure. One or more inositol/IP-6 composition may be administered simultaneously, or different inositol/IP-6 compositions may be administered at different times.

When multiple acute exposures are anticipated, the inositol/IP-6 compositions may be administered multiple times. For example, if fire or rescue personnel must enter contaminated areas multiple times, inositol/IP-6 compositions may be administered prior to each exposure. Preferably, an about 24 hour period separates administration of inositol/IP-6 compounds and the radiation exposure. More preferably, the administration of inositol/IP-6 compounds and the radiation exposure is separated by about 6 to 18 hours, and even more preferably the administration of inositol/IP-6 compounds and the radiation exposure is separated by about 2-3 hours. It is also contemplated that a worker in a nuclear power plant may be administered an effective amount of inositol/IP-6 compositions prior to beginning each shift to reduce or eliminate the effects of exposure to ionizing radiation.

If a subject is anticipating chronic exposure to ionizing radiation, the inositol/IP-6 compositions may be administered periodically throughout the duration of anticipated exposure. For example, a nuclear power plant worker or a soldier operating in a forward area contaminated with radioactive fallout may be given inositol/IP-6 compositions every 24 hours, preferably every 6-18 hours, and even more preferably every 3-6 hours in order to mitigate the effects of radiation damage. Likewise, inositol/IP-6 compositions may be periodically administered to civilians living in areas contaminated by radioactive fallout until the area is decontaminated or the civilians are removed to a safer environment.

If a subject is anticipating prolonged exposure to the sun, and therefore to its ultraviolet radiation, the inositol/IP-6 compounds of the present invention may be administered prior to the anticipated exposure to prevent acute adverse effects, i.e., sunburn. Preferably, the inositol/IP-6 compositions are administered at least about 2-12 hours prior to exposure. Even more preferably, administration is performed at least about 2-6 hours prior to exposure, and more preferably yet between about 2-3 hours prior to exposure. Administration may also preferably be performed immediately prior to the exposure. Administration is preferably topical in the form of a lotion, cream, or gel. Even more preferably, the topical form will include a sun screen or sun block, and may further include moisturizers, colorants, perfumes, biocides, and the like as one skilled in the art would find desirable. The subject may also, in addition to or even in place of topical administration, be administered oral compositions of the present invention. Preferably, inositol/IP-6 compositions may be dissolved in water and consumed 2-12, 2-6, or most preferably 2-3 hours prior to sun exposure. Preferably, the inositol/IP-6 compositions are re-administered at least about every 12 hours, more preferably about every 8 hours, and even more preferably at least about every 2-6 hours.

As used herein, “administered” means the act of making the inositol/IP-6 compounds available to the subject such that a pharmacologic effect of radioprotection or remediation is realized. This pharmacologic effect may manifest as the absence of expected physiologic or clinical symptoms at a certain level of radiation exposure. One skilled in the art may readily determine the presence or absence of radiation-induced effects by well-known laboratory and clinical methods. The inositol/IP-6 compound may thus be administered by any route which is sufficient to bring about the desired radioprotective effect in the patient. Routes of administration include, for example topical or enteral (e.g., oral, rectal, intravaginal, intranasal, etc.) and parenteral administration. Parenteral administration includes, for example, intravenous, intramuscular, intraarterial, intraperitoneal, intravesicular (e.g., into the bladder), intradermal, or subcutaneous administration. Also contemplated within the scope of the invention is the instillation of drug in the body of the patient in a controlled formulation, with systemic or local release of the drug to occur at a later time. For example, a depot of inositol/IP-6 compound maybe administered to the patient more than 24 hours before the administration of radiation. Preferably, at least a portion of the inositol/IP-6 is retained in the depot and not released until an about 6-18 hour window prior to the radiation exposure, and even more preferably until about 2-3 hours prior to radiation exposure.

The inositol/IP-6 compound may be administered in the form of a pharmaceutical composition comprising one or more inositol/IP-6 compounds in combination with one or more pharmaceutically or pharmacologically acceptable carriers. As noted above, the inositol/IP-6 compound in such formulations may comprise from 0.01 to about 100 weight percent. By “pharmaceutically” or “pharmacologically acceptable carrier” is meant any carrier, diluent or excipient which is compatible with the other ingredients of the formulation and is not deleterious to the subject. It is within the skill in the art to formulate appropriate pharmaceutical compositions with inositol/IP-6 compositions.

For example, the inositol/IP-6 compositions may be formulated into pharmaceutical compositions according to standard practices in the field of pharmaceutical preparations. See Alphonso Gennaro, ed., Remington's Pharmaceutical Sciences, 18th Ed., (1990) Mack Publishing Co., Easton, Pa. Suitable pharmaceutical compositions include, for example, tablets, powders, capsules, solutions (especially parenteral solutions), troches, suppositories, creams, lotions, gels, or suspensions.

The active ingredient may be administered at once or may be divided into a number of smaller doses to be administered simultaneously or at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The compound may be suspended in micronized or other suitable form or may be derivatized to produce a more soluble active product or to produce a prodrug, or where the compound is a prodrug, to use the active form. The form of the resulting mixture depends upon a number of factors, including the intended mode of administration and the solubility of the compound in the selected carrier or vehicle. The effective concentration is sufficient for ameliorating the adverse health effects of ionizing radiation exposure and may be empirically determined.

For parenteral administration, the inositol/IP-6 compound may be mixed with a suitable carrier or diluent such as water, an oil, saline solution, aqueous dextrose (glucose) and related sugar solutions, cyclodextrans or a glycol such as propylene glycol or polyethylene glycol. Solutions for parenteral administration preferably contain a pharmaceutically or pharmacologically acceptable, water soluble salt of the inositol/IP-6 compound. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol.

For oral administration, the inositol/IP-6 may be combined with one or more solid inactive ingredients for the preparation of tablets, capsules, or other suitable oral dosage forms. For example, the active agent may be combined with carboxymethylcellulose calcium, magnesium stearate, mannitol and starch, and then formed into tablets by conventional tableting methods.

The specific dose and schedule of inositol/IP-6 composition administration to obtain the radioprotective benefit will, of course, be determined by the particular circumstances of the individual patient including, the size, weight, age and sex of the patient, the nature and stage of the disease being treated, the aggressiveness of the disease, and the route of administration, and the specific toxicity of the radiation. For example, a daily dosage of from about 0.01 to about 150 mg/kg/day may be utilized, more preferably from about 0.05 to about 50 mg/kg/day. Particularly preferred are doses from about 1.0 to about 40.0 mg/kg/day, for example, a dose of about 30 mg/kg/day. The dose may be given over multiple administrations, for example, two administrations of 15 mg/kg. Higher or lower doses are also contemplated.

The inositol/IP-6 compositions may take the form of pharmaceutically acceptable salts. The term “pharmaceutically acceptable salts” embraces salts commonly used to form alkali metal salts and to form addition salts of free acids or free bases. The nature of the salt is not critical, provided that it is pharmaceutically-acceptable. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, galactaric and galacturonic acid. Suitable pharmaceutically acceptable base addition salts include metallic salts made from calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding inositol/IP-6 compound by reacting, for example, the appropriate acid or base with the inositol/IP-6 compound.

### EXAMPLE 1

### Improved Cell Viability by Administering Inositol/IP-6 Compounds Following Radiation Standard Procedures:

The human keratinocyte (HaCaT) cells were grown in Dulbecco's Modified Eagle's Medium containing 7 mg/L of inositol (38.8 μM), supplemented with 10% heat-inactivated fetal bovine serum, 1% L-glutamine, and 1% antibiotic (penicillin, streptomycin). Cells were maintained at 37° C. and 5% CO₂. Na-IP-6 was diluted from a 100 mM stock solution to the required concentrations (0.05-2.0 mM) using cell culture medium as the diluent. UVB intensities of 15, 30, 60 and 120 mJ/cm² were used, which was obtained by varying cell exposure time to UVB light (80% of light output is in 290-320 nm UVB range).

HaCaT cells were grown to approximately 80-90% confluency. 100 μL of 1×10⁴ cells/mL HaCaT cells were seeded into each well of four 96-well plates. Cells were treated with IP-6 (0-2.0 mM), and on days 0, 1, 2, and 3, 100 μL of 1 mg/mL of aqueous MTT dissolved in DMEM media was added to each well and plates were placed into the incubator (37° C. and 5% CO₂) for 4 hours. 150 μL of DMSO was then added to dissolve the formazan products. The absorbance was read at 540 nm using an EL Ultra Micro-plate Reader. Results were recorded as mean absorbance for each set of groups. For Plating Efficiency Assay, HaCaT cells were plated at 500 cells/well in 12-well tissue culture dishes. IP-6 at concentrations between 0-2.0 mM was added to the respective wells in duplicate. The cells were then incubated at 37° C. and 5% CO₂ for 7 days.

Following this incubation period, the control and treated colonies were washed with PBS 1× (pH 7.4), fixed with 4.0% formaldehyde and stained with 0.5% of aqueous crystal violet. The number of colonies was counted using an inverted microscope. Results were recorded as the mean number of colonies for each group. Statistical analysis: each experiment was performed at least twice, and expressed as the mean ± standard deviations, which was calculated using Excel software. The Student's t-test was used to compare control and experimental groups, and differences were considered significant with p value <0.05. The aforementioned procedures were used in Examples 1-6.

To determine if IP-6 protects HaCaT cells from UVB-induced injury, The MTT assay was performed as done above. 50 μL of 2×10⁴ cells/mL in DMEM media without phenol red was seeded into each well of five 96 well plates and incubated (37° C. and 5% CO₂) for 24 hours. Cells were then exposed to 0, 15, 30, 60 or 120 mJ/cm² UVB intensities (UVB broad band lamps, bank of 4 (FS40T12/UVB 4 ft)). Immediately afterwards the UVB-exposed cells were treated with IP-6 (0-2.0 mM) in 50 μL of cell media. The cells were then placed in the incubator at 37° C. and 5% CO₂ and addition of MTT was performed as above. Results were recorded as the mean absorbance for each set of groups.

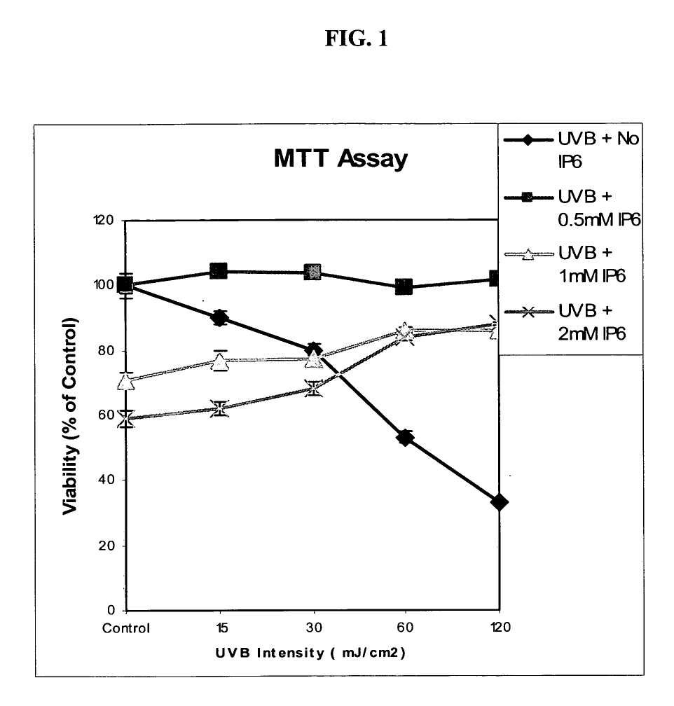

UVB radiation caused a dose dependent decrease in viability and proliferation of HaCaT cells 24 hours after exposure as compared to the non-exposed control group, p<0.05. Significance was shown by increase in cell viability with increasing concentration of IP-6 24 hours after UVB exposure, p<0.001 (FIG. 1). While the normal trend is towards a decrease in cell viability with higher concentration of IP-6, the results show a reverse trend with an increase in cell viability as the UVB intensity increases.

### EXAMPLE 2

### The Effects of IP-6 and UVB Radiation on Attached Cells

5×10⁴ HaCaT cells were seeded into each well of four 6-well tissue culture plates and incubated at 37° C. and 5% CO₂ for 24 hours. One hour before UVB irradiation, two plates were treated with IP-6 (0 and 0.1 mM), one for IP-6 pre-treatment and the other for IP-6 pre-treatment and post-treatment. All four plates were then washed twice with PBS 1×, and a small amount of PBS 1× was added to the wells, which were irradiated at 30 mJ/cm². After UVB exposure, PBS was removed from the wells and DMEM media was added to each well. IP-6 (0.05 and 0.1 mM) was then added to the plates labeled no UVB exposure, IP-6 post-treatment, and pre-treatment and post-treatment. The cells were then incubated (37° C. and 5% CO₂) for 18 hours. Following incubation, the cells were washed 4 times with PBS 1× (pH 7.4), then fixed with 4.0% formaldehyde for 15 minutes, and stained with 0.5% aqueous crystal violet for at least 5 minutes. Excess crystal violet was washed from the wells and the plates were left to dry. The dried crystal violet residue in each well was then dissolved in 500 μL of 30% acetic acid. The absorbance was read at 595 nm in triplicate in a 96-well plate using an EL Ultra Micro-plate Reader.

Additionally, 5×10⁴ HaCaT cells were plated into each well of ten 6-well plates as was done above. Before UVB irradiation, cells were washed twice with PBS 1×, and a small amount of PBS 1× was added to the wells. Cells were then exposed to no UVB, or 15, 30, 60, or 120 mJ/cm² intensities. Following exposure, cell media was added to the wells and cells were treated with IP-6 (0-2.0 mM). The cells were then incubated at 37° C. and 5% CO₂. 18 hours after UVB exposure, cells were fixed with 4% formaldehyde, stained with 0.5% aqueous crystal violet and dissolved with 30% acetic acid as was done above. The absorbance was read in triplicate at 595 nm.

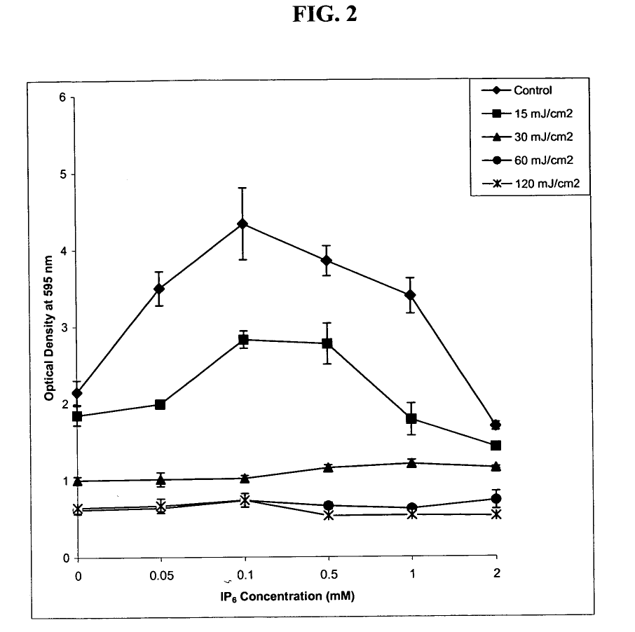

Exposure of HaCaT cells to different UVB intensities showed a significant dose-dependent decrease in cell attachment with increasing UVB intensities as compared to the non-exposed control group 18 hours after UVB exposure, p<0.001. In order to determine the effect of both IP-6 and UVB on cell attachment, different concentrations of IP-6 with different UVB intensities were used. A significant increase in HaCaT cell attachment with increasing concentrations of IP-6 at a UVB intensity of 30 mJ/cm², as compared to cells irradiated at 30 mJ/cm² but not treated with IP-6 was observed (significant at p<0.01). Both 60 and 120 mJ/cm² VB intensities showed a decrease in cell attachment as compared to their respective control groups, that is, cells exposed to either 60 or 120 mJ/cm² UVB and no IP-6. Groups exposed to 15 mJ/cm² UVB showed an increase in cell attachment for lower concentrations of IP-6, and a decrease in cell attachment for higher IP-6 concentrations, that is, 1.0 and 2.0 mM, as compared to the non-IP-6 treated group. The trend shown by cells exposed to 15 mJ/cm² UVB was comparable to the group exposed to different IP-6 concentrations but no UVB exposure (FIG. 2).

### EXAMPLE 3

### Effect of IP-6 and UVB Radiation on Apoptosis of HaCaT Cells.

HaCaT cells were plated in 60 mm tissue culture dishes for 24 hours. The cells were then either not exposed, or exposed, to 30 mJ/cm² UVB radiation and treated immediately with 0, 0.5 mM, or 1.0 mM IP-6. Cells were harvested 6 and 18 hours after UVB exposure. 5 μL of RNase (DNase-free) was added to 10⁶ cells/mL. The cell suspension was incubated at 37° C. for 30 minutes. The suspension was then chilled on ice (2-8° C.). 100 μL of PI was added to the cell suspension (Cellular DNA flow cytometric analysis kit, Roche Diagnostics, Indianapolis, Ind.). DNA quantitation was performed on the same day by flow cytometry using FACS.

At 6 hours following 30 mJ/cm² UvB radiation, there was a significant increase in the G1 phase and a significant decrease in the S phase as compared to the non-UVB exposed control group, p<0.001. However, there was no significant difference in the G2M phase between the exposed and non-exposed groups. 18 hours after UVB exposure, there was a significant increase in the G1 phase and a significant decrease in the S phase as compared to the non-exposed group, and even after 18 hours, UVB radiation had no significant effect on the G2M phase of the cell cycle. There were no time-dependent differences between 6 and 18 hour exposures and the effect of IP-6 on different phases of the cell cycle.

Both 0.5 mM and 1.0 mM IP-6 treatments after UVB exposure showed similar results 6 hours after exposure to UVB radiation. The G2M phase 18 hours after UVB exposure followed by immediate treatment with 1.0 mM IP-6 was comparable to results seen 18 hours after treatment with 0.5 mM IP-6.

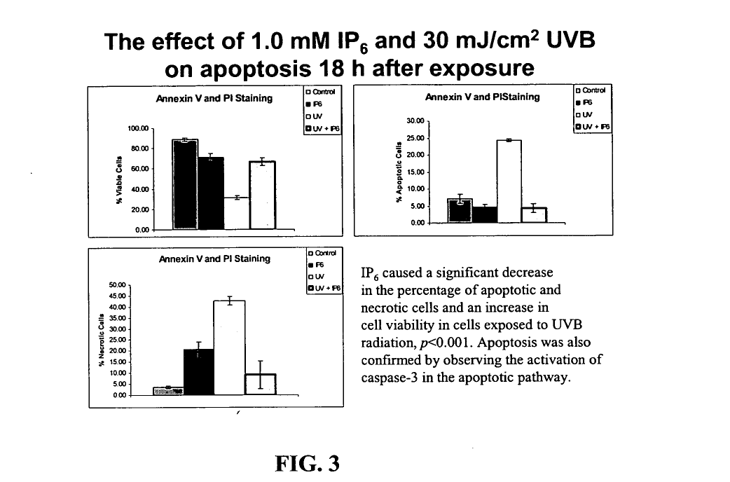

0.5 mM IP-6 treatment on cells exposed to UVB showed a significant decrease in the percentage of both apoptotic and necrotic cells and a significant increase in the percentage of viable cells, as compared to groups exposed to UVB radiation but not treated with IP-6 (FIG. 3). 1.0 mM IP-6 treatment showed similar results as compared to 0.5 mM IP-6 treatment, however the effects were much more pronounced at higher concentrations. IP-6 was therefore able to cause a dose-dependent decrease in both apoptotic and necrotic cells, and an increase in viable cells, when exposed to UVB radiation, as compared to the group exposed to UVB but not treated with IP-6, p<0.001.

Comparable results are obtained when the experiment is repeated using MCF-10A (immortalized normal breast cell line) and normal human peripheral lymphocytes.

### EXAMPLE 4

### Effect of IP-6 and UVB Radiation on Caspase-3 Activation of HaCaT Cells.

HaCaT cells were plated in 60 mm tissue culture dishes for 24 hours. The cells were either exposed to 30 mJ/cm² UVB radiation or not exposed. Immediately following UVB exposure, cells were treated with 1.0 mM of IP-6. Eighteen hours after UVB exposure, cell lysates obtained from 1×10⁶ HaCaT cells were prepared in hypotonic cell lysis buffer (25 mM HEPES (pH 7.5), 5 mM MgCl₂, 5 mM EDTA, 5 mM DTT, 2 mM PMSF, 10 μg/mL Pepstatin A, and 10 μg/mL Leupeptin). 10 μL of sample was then combined with caspase assay buffer (312.5 mM HEPES (pH 7.5), 31.25% sucrose, 0.3125% CHAPS (3-[(3-cholamido-propyl)-dimethyl ammonio]-1 propane-sulfonate, 2% DMSO, 10 mM DTT) containing 50 μM caspase-3 substrate, Ac-DEVD-AMC, in white 96 well microtiter plates. Negative control wells included 2 μL of 2.5 mM specific peptide inhibitor of caspase-3 (Ac-DEVD-CHO). The plate was incubated at 30° C. for 1 hour. The free reaction product, AMC was then measured using a fluorescent plate reader with an excitation wavelength of 360 nm and an emission wavelength of 460 nm. Caspase-3 activity was reported as relative fluorescent intensity of AMC.

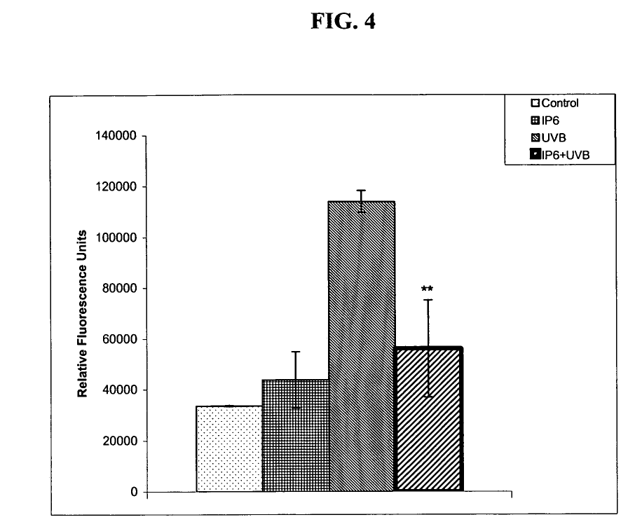

The effect of IP-6 on apoptosis of HaCaT cells was determined using 1.0 mM IP-6 treatment, 18 hours after exposure to 30 mJ/cm² UVB radiation. Result shows that IP-6 caused an increase in activated caspase-3 activity as compared to the control group. UVB exposure also caused an increase in activated caspase-3 activity. However, in the presence of 1.0 mM IP-6, activated caspase-3 activity was significantly lowered as compared to the group exposed to UVB radiation but not treated with IP-6, p<0.01 (FIG. 4).

Comparable results are obtained when the experiment is repeated using MCF-10A (immortalized normal breast cell line) and normal human peripheral lymphocytes.

### EXAMPLE 5

### IP-6±Inositol as Radioprotectant

Human keratinocyte HaCaT cells were exposed to UVB 30 mJ/cm² (2 minutes 10 seconds); non-exposed cells served as control. The cells were then treated with IP-6, inositol, IP-6+inositol, and untreated (control) and placed in the incubator for 18 hours. The cells that remained attached to the wells are live (protected from UVB damage); the amount of attached cells is measured by dissolving them in acetic acid and measuring the optical absorbance of the solution. Cells treated with 1:1 molar ratio of IP-6 and inositol showed the best attachment.

Comparable results are obtained when the experiment is repeated using MCF-10A (immortalized normal breast cell line) and normal human peripheral lymphocytes.

### EXAMPLE 6

### Effect of IP-6 Inositol as Radioprotectant In Vivo

Six-week-old pathogen-free female SKH-1 female mice (Charles River Laboratory, Wilmington, Mass.), were irradiated 3 times a week, initially with 1.5 kJ/m² dose and escalating weekly by 1.5 kJ/m² to a final dose of 7.5 kJ/m²; each session lasted approximately 10 minutes for 23 weeks. Animals were fed with AIN-76A diet (Harlan Teklad, #170481) that does not contain IP-6. About 100 mg of IP-6+/−Inositol was applied on the dorsal surface topically in skin cream as 4% K-IP-6, 1% inositol, or 4% K-IP-6+1% inositol. An additional group received Na-IP-6+inositol at 1:1 molar ratio in drinking water to see if orally administered IP-6+inositol would provide similar protection. The animals were observed daily and the appearance of tumors in the form of papillomas was monitored and counted. Results show that animals that were treated with K-IP-6 in skin cream had no tumors as opposed to the cream without IP-6; IP-6+inositol in cream showed a 60% reduction in tumors and even more interestingly animals who received IP-6+inositol in drinking water had a 78.6% reduction in UVB-induced skin tumor incidence.

Furthermore, treated animals show ameliorated radiation-induced skin damage in comparison to control animals, with reduced or absent reddening of the skin, ulceration, and/or blistering.

### EXAMPLE 7 (PROPHETIC)

### Radioprotective Effects of Inositol/IP-6 Compositions on Cultured Normal Cells

HFL-1 cells, which are normal diploid lung fibroblasts, are plated into 24 well dishes at a cell density of 3,000 cells/10 mm² in DMEM completed with 10% fetal bovine serum and antibiotics. The inositol/IP-6 test compounds are added to the cells 24 hours later in select concentrations from 100 to 500 micromolar, inclusive, in water. Control cells are treated with water alone. The cells are exposed to the test compound or water for 24 hours. The cells are then irradiated with either 10 Gy (gray) or 15 Gy of ionizing radiation (IR) using a J. L. Shepherd Mark I, Model 30-1 Irradiator equipped with ¹³⁷cesium as a source.

After irradiation, the medium on the test and control cells is removed and replaced with fresh growth medium without the test compounds or additional water. The irradiated cells are incubated for 96 hours and duplicate wells are trypsinized and replated onto 100 mm2 tissue culture dishes. The replated cells are grown under normal conditions with one change of fresh medium for 3 weeks. The number of colonies from each 100 mm² culture dish, which represents the number of surviving cells, is determined by staining the dishes as described below.

To visualize and count the colonies derived from the clonal outgrowth of individual radioprotected cells, the medium is removed and the plates are washed one time with room temperature phosphate buffered saline. The cells are stained with a 1:10 diluted Modified Giemsa staining solution (Sigma) for 20 minutes. The stain is removed, and the plates are washed with tap water. The plates are air dried, the number of colonies from each plate are counted and the average from duplicate plates is determined.

Radioprotective activity for the inositol/IP-6 compounds is seen.

Comparable results are obtained when the experiment is repeated using MCF-10A (immortalized normal breast cell line) and normal human peripheral lymphocytes.

### EXAMPLE 8 (PROPHETIC)

### Treatment of Cultured Tumor Cells with Inositol/IP-6 Compositions

In order to address the effect of the inositol/IP-6 compositions on tumor cell killing by ionizing irradiation under conditions that are protective for normal fibroblasts, the following experiments are conducted. DU145 cells, an androgen negative prostate carcinoma cell line, are plated in 6 well dishes at a cell density of 1.0×10⁵ cells per 35 mm2 in DMEM completed with 10% fetal bovine serum and antibiotics. Inositol/IP-6 compositions (0.5 mM, 1.0 mM, 2.5 mM, 5.0 mM, 10.0 mM and 20.0 mM) in water are added separately to the cells 24 hours later. Control cells receive water alone. The plates are incubated for 2-4 hours and the cells are irradiated with either 5 Gy or 10 Gy of radiation.

After irradiation, the medium is removed and replaced with fresh medium without the test compound. The cells are incubated for 96 hours and the number of viable cells is determined by trypan blue exclusion. The average number of viable cells from duplicate wells is determined.

The addition of the inositol/IP-6 composition that induce radioprotection in normal human lung fibroblasts does not reduce the killing activity of ionizing radiation on the tumor cell line. A small but consistent additive affect on cell killing of the tumor cells is also seen. The radioprotective effect of the inositol/IP-6 compound is specific for normal tissue, and does not interfere with the killing of tumor cells by IR when the tumor cells are treated with the test compounds as a 2-4 hour pulse prior to irradiation.

Comparable results are obtained when the experiment is repeated using MCF-10A (normal immortalized breast cell) and normal human peripheral lymphocytes.

### EXAMPLE 9 (PROPHETIC)

### Protection of Mice from Radiation Toxicity by Pre-Treatment with Inositol/IP-6 Compositions

C57 black mice aged 10-12 weeks (Taconic) are divided into two treatment groups of 10 mice each. One group receives intraperitoneal injections of 200 micrograms inositol/IP-6 compositions dissolved in water (a 10 mg/Kg dose, based on 20 g mice) 18 and 6 hours before irradiation with 8 Gy gamma radiation. A second group receives intraperitoneal injections of 600 micrograms inositol/IP-6 compositions dissolved in water (a 30 mg/Kg dose, based on 20 g mice) 18 and 6 hours before irradiation with 8 Gy gamma radiation. A control group of 16 animals received 8 Gy gamma radiation alone. Mortality of control and experimental groups is assessed for 40 days after irradiation.

By day 20 post-irradiation, the control mice exhibit a maximum mortality rate of 80%; the 8 Gy dose of gamma radiation is thus considered an LD₈₀ dose. By contrast, only about 50% of the first group and about 30% of the second group mice are dead at day 20 after receiving the LD₈₀ radiation dose. By day 40, a maximum mortality rate of approximately 60% is reached in the first group, and a maximum mortality rate of approximately 50% is reached in the second group. Radiation toxicity in mice is substantially reduced by pretreatment with inositol/IP-6 compositions.

### EXAMPLE 10 (PROPHETIC)

### Radioprotective Effect of Inositol/IP-6 Compositions in Mice when Given after Radiation Exposure

C57 B6/J mice age 10-12 weeks (Taconic) are divided into two treatment groups of 10 and 9 mice, respectively. One group receives intraperitoneal injections of 200 micrograms inositol/IP-6 compositions dissolved in water (a 10 mg/Kg dose, assuming 20 g mice) 18 and 6 hours before irradiation with 8 Gy gamma radiation. A second group receives an intraperitoneal injection of 600 micrograms inositol/IP-6 compositions dissolved in water (a 30 mg/Kg dose, based on 20 g mice) 15 minutes after irradiation with 8 Gy gamma radiation. A control group of 16 animals receives 8 Gy gamma radiation alone. Mortality of control and experimental groups is assessed for 40 days after irradiation.

Treatment of mice with inositol/IP-6 compositions after irradiation results in significant delay in radiation-induced mortality as compared with the control animals. While the radioprotective effects that inositol/IP-6 compositions afford by post-irradiation treatment is not as great as those seen with pre-irradiation treatment, post-irradiation treatment with inositol/IP-6 compositions is nonetheless effective in mitigating the effects of radiation toxicity.

### EXAMPLE 11 (PROPHETIC)

### Effect of Exposure to Ionizing Radiation on Normal and Malignant Hematopoietic Progenitor Cell Growth after Pretreatment with Inositol/IP-6 Compositions

The effect of ionizing radiation on normal and malignant hematopoietic progenitor cells which are pretreated with inositol/IP-6 compositions is determined by assessing cloning efficiency and development of the pretreated cells after irradiation.

To obtain hematopoietic progenitor cells, human bone marrow cells (BMC) or peripheral blood cells (PB) are obtained from normal healthy, or acute or chronic myelogenous leukemia (AML, CML) volunteers by Ficoll-Hypaque density gradient centrifugation, and are partially enriched for hematopoietic progenitor cells by positively selecting CD34⁺ cells with immunomagnetic beads (Dynal A. S., Oslo, Norway). The CD34⁺ cells are suspended in supplemented alpha medium and incubated with mouse anti-HPCA-I antibody in 1:20 dilution, 45 minutes, at 4° C. with gentle inverting of tubes. Cells are washed three times in supplemented alpha medium, and then are incubated with beads coated with the Fc fragment of goat anti-mouse IgG₁ (75 microliters of immunobeads/10⁷ CD34⁺ cells). After 45 minutes of incubation (4° C.), cells adherent to the beads are positively selected using a magnetic particle concentrator as directed by the manufacturer.

2×10⁴ CD34⁺ cells are incubated in 5 ml polypropylene tubes (Fisher Scientific, Pittsburgh, Pa.) in a total volume of 0.4 ml of Iscove's modified Dulbecco's medium (IMDM) containing 2% human AB serum and 10 mM Hepes buffer. Inositol/IP-6 compositions are added to the cells; for example, inositol/IP-6 compositions in three different concentrations (5.0 mM, 10.0 mM and 20.0 mM) in water are added separately to the cells. Control cells receive water alone. The cells are incubated for 20-24 hours and irradiated with 5 Gy or 10 Gy of ionizing radiation.

Immediately after irradiation, the medium is removed and replaced with fresh medium without the test compound. Twenty-four hours after irradiation, the treatment and control cells are prepared for plating in plasma clot or methylcellulose cultures. Cells (1×10⁴ CD34⁺ cells per dish) are not washed before plating.

Assessment of the cloning efficiency and development of the treated hematopoietic progenitor cells are carried out essentially as reported in Gewirtz et al., Science 242, 1303-1306 (1988). Substantial beneficial effects in the treated samples as compared to the controls are seen.

### EXAMPLE 12 (PROPHETIC)

### Bone Marrow Purging with Ionizing Radiation after Pretreatment with Inositol/IP-6 Compositions

Bone marrow is harvested from the iliac bones of a subject under general anesthesia in an operating room using standard techniques. Multiple aspirations are taken into heparinized syringes. Sufficient marrow is withdrawn so that the subject will be able to receive about 4×10⁸ to about 8×10⁸ processed marrow cells per kg of body weight. Thus, about 750 to 1,000 ml of marrow is withdrawn. The aspirated marrow is transferred immediately into a transport medium (TC-199, Gibco, Grand Island, N.Y.) containing 10,000 units of preservative-free heparin per 100 ml of medium. The aspirated marrow is filtered through three progressively finer meshes to obtain a cell suspension devoid of cellular aggregates, debris, and bone particles. The filtered marrow is then processed further into an automated cell separator (e.g., Cobe 2991 Cell Processor) which prepares a “buffy coat” product, (i.e., leukocytes devoid of red cells and platelets). The buffy coat preparation is then placed in a transfer pack for further processing and storage. It may be stored until purging in liquid nitrogen using standard procedures. Alternatively, purging can be carried out immediately, then the purged marrow may be stored frozen in liquid nitrogen until it is ready for transplantation.

The purging procedure is carried out as follows. Cells in the buffy coat preparation are adjusted to a cell concentration of about 2×10⁷/ml in TC-199 containing about 20% autologous plasma. Inositol/IP-6 compositions, for example, 1-2 millimolar of inositol/IP-6 compositions in water or 10-20 millimolar inositol/IP-6 compositions in water are added to the transfer packs containing the cell suspension and incubated in a 37° C. water bath for 20-24 hours with gentle shaking. The transfer packs are then exposed to 5-10 Gy ionizing radiation. Recombinant human hematopoietic growth factors, e.g., rH IL-3 or rH GM-CSF, may be added to the suspension to stimulate growth of hematopoietic neoplasms and thereby increase their sensitivity to ionizing radiation.

The cells are then either frozen in liquid nitrogen or washed once at 4° C. in TC-199 containing about 20% autologous plasma. Washed cells are then infused into the subject. Care is taken to work under sterile conditions wherever possible and to maintain scrupulous aseptic techniques at all times.

### EXAMPLE 13 (PROPHETIC)

### Protective Effect of IP-6 Against UVB-Induced Cell Damage and Injury in Normal Mammary Epithelial Cells

The non-transformed, normal human mammary epithelial cells MCF-10A cells are utilized to demonstrate the protective effect of insitol/IP-6 compositions on UVB-induced cell damage by measuring cell viability and cell attachment. The MCF-10A (spontaneously immortalized, non-tumorigenic human mammary epithelial cell line with non-mutated p53) is maintained in F-12/DMEM supplemented with 5% horse serum (Invitrogen Gibco, Carlsbad, CA), 20 ng/mL EGF (Upstate Biotechnology Incorporated, Lake Placid, N.Y.), 10 μg/mL insulin (Biofluids, Rockville, Md.), and 500 ng/mL hydrocortisone. Cells are maintained at 37° C. and 5% CO₂. Na-IP-6 is diluted from a 100 mM stock solution to the required concentrations (0.05-2.0 mM) using cell culture medium as the diluent. UVB intensities of 15, 30, 60 and 120 mJ/cm² are used, which will be obtained by varying cell exposure time to UVB light (80% of light output is in 290-320 nm UVB range).

MCF-10A cells are grown to approximately 80-90% confluency. 100 μL of 1×10⁴ cells/mL of MCF-10A cells are seeded into each well of four 96-well plates. To assess viability using the MTT-based cytotoxicity assay, cells are treated with IP-6 (0-2.0 mM), and on days 0, 1, 2 and 3, 100 μL of 1 mg/mL of aqueous MTT dissolved in DMEM media are added to each well and plates will placed into the incubator (37° C. and 5% CO₂) for 4 hours. 150 μL of DMSO is added to dissolve the formazan products. The absorbance is read at 540 nm using an EL Ultra Micro-plate Reader. Results are recorded as mean absorbance for each set of groups. To determine if IP-6 protects MCF-10A cells from UVB-induced injury, the MTT assay is performed as described above. 50 μL of 2×10⁴ cells/mL in DMEM media without phenol red are seeded into each well of five 96 well plates and incubated (37° C. and 5% CO₂) for 24 hours. Cells are then exposed to 0, 15, 30, 60 or 120 mJ/cm² UVB intensities ((UVB broad band lamps, bank of 4 (FS40T12/UVB 4 ft)). Immediately afterwards, the UVB-exposed cells are treated with IP-6 (0-2.0 mM) in 50 μL of cell growth media. The cells are then placed in the incubator at 37° C. and 5% CO₂ and the MTT assay is performed as above. Results are recorded as the mean absorbance for each set of groups.

Attachment of cells is used as an indirect indicator of cell viability for anchorage-dependent cells that normally grow in monolayers because viable cells remain attached and dying/dead cells are detached. To determine the effects of inositol/IP-6 compositions and UVB radiation on attached cells, 5×10⁴ MCF-10A cells are seeded into each well of four 6-well tissue culture plates and incubated at 37° C. and 5% CO₂ for 24 hours. One hour before UVB irradiation, two plates are treated with IP-6 (0 and 0.1 mM), one for IP-6 pre-treatment and the other for IP-6 pre-treatment and post-treatment. All four plates are then washed twice with PBS IX, and a small amount of PBS 1× is added to the wells, which are irradiated at 30 mJ/cm². After UVB exposure, PBS is removed from the wells and DMEM media is added to each well. IP-6 (0.05 and 0.1 mM) is then added to the plates labeled no UVB exposure, IP-6 post-treatment, and pre-treatment and post-treatment. The cells are incubated (37° C. and 5% CO₂) for 18 hours. Following incubation, the cells are washed 4 times with PBS 1× (pH 7.4), then fixed with 4.0% formaldehyde for 15 minutes and stained with 0.5% aqueous crystal violet for at least 5 minutes. Excess crystal violet is washed from the wells and the plates are left to dry. The dried crystal violet residue in each well is then dissolved in 500 μL of 30% acetic acid. The absorbance is read at 595 nm in triplicate in a 96-well plate using an EL Ultra Micro-plate Reader.

Additionally, 5×10⁴ MCF-10A cells are plated into each well of ten 6-well plates as described above. Before UVB irradiation, cells are washed twice with PBS 1×, and a small amount of PBS 1× is added to the wells. Cells are then exposed to no UVB or 15, 30, 60, or 120 mJ/cm² intensities. Following exposure, cell media is added to the wells and cells are treated with IP-6 (0-2.0 mM). The cells are incubated at 37° C. and 5% CO₂. 18 hours after UVB exposure, cells are fixed with 4% formaldehyde, stained with 0.5% aqueous crystal violet, and dissolved with 30% acetic acid as described above. The absorbance is read in triplicate at 595 nm.

Radioprotective effects of the inositol/IP-6 compositions are observed.

### EXAMPLE 14 (PROPHETIC)

### Effect of Ionizing Radiation and IP-6 on Normal Peripheral Mononuclear Cells

The effect of exposure to ionizing radiation and IP-6 on the viability of normal peripheral mononuclear cells (PBMCs) is assessed by their ability to proliferate and form colonies. A number of assays have been developed in semisolid media to detect T cell colony-forming cells (T-CFCs). These T-CFCs have been used to determine the number of T cell progenitors and to study the proliferation and differentiation capacity of peripheral blood and bone marrow T lymphocytes in normal subjects and in patients with variety of pathological conditions. T cell colonies are generated from the PBMCs of 10-20 healthy volunteers. The effect is observed on spontaneous and induced T cell colonies. PBMCs cultured in methylcellulose in the absence of added growth factors (PHA and IL-2) form spontaneous T cell colonies; in the presence of growth factors they form induced T cell colonies.

PBMCs are separated on Ficoll-Hypaque (Pharmacia, Upsalla, Sweden). Interphase cells are washed twice with PBS and resuspended in growth medium, α-modified Eagle's Medium (α-MEM) (Invitrogen Gibco, Carlsbad, Calif.). Their viability, as tested by trypan blue dye exclusion, will always be >90%. Cells can be further separated on the basis of rosette formation or fractionated using immuno-magnetic beads as described in Example 11. Fractionated or unfractionated PBMC cells (5×10⁵/ml cells/mL) are seeded in 0.8% methylcellulose (Fluka Chemie AG, Buchs, Switzerland) in α-MEM supplemented with 20% FCS, 2 mM glutamine, and antibiotics in the presence (induced T colonies) of PHA (1%; vol/vol) and 10 U/mL of human rIL-2 (Biogen, Geneva, Switzerland), and in the absence of PHA and rIL-2 (spontaneous T cell colonies). 0.1 mL of cell-containing methylcellulose preparation are seeded per well in 96-well flat-bottom microtest plates and incubated at 37° C. and 5% CO₂ in air for 5-7 days. Aggregates containing at least 50 cells are counted under an inverted microscope as colonies.

Peripheral blood and isolated PBMCs are exposed to 5 Gy or 10 Gy of ionizing irradiation. The protective effect of inositol/IP-6 compositions is determined by assessing the colony forming ability of irradiated PBMC T-CFCs either pretreated with IP-6 (0-2.0 mM) 1 hour prior to irradiation or in the presence of inositol/IP-6 compositions during the incubation and colony formation process.

Substantial protective effects of inositol/IP-6 compositions on the test cells are observed.

### Topical Compositions

Topical products occur in a variety of forms, including solids, liquids, suspensions, semisolids (such as creams, gels, pastes or “sticks”), powders or finely dispersed liquids such as sprays or mists. Examples of topical products commonly classified as “cosmetics” include skin care products such as creams, lotions, moisturizers and “treatment cosmetics” such as exfoliants and/or skin cell renewal agents; fragrances such as perfumes and colognes, and deodorants; shaving-related products such as creams, “bracers” and aftershaves; depilatories and other hair removal products; skin cleansers, toners and astringents; pre-moistened wipes and washcloths; tanning lotions; bath products such as oils; eye care products such as eye lotions and makeup removers; foot care products such as powders and sprays; skin colorant and make-up products such as foundations, blushes, rouges, eye shadows and liners, lip colors and mascaras; lip balms and sticks; hair care and treatment products such as shampoos, conditioners, colorants, dyes, bleaches, straighteners and permanent wave products; baby products such as baby lotions, oils, shampoos, powders and wet wipes; feminine hygiene products such as deodorants and douches; skin or facial peels applied by dermatologists or cosmeticians; and others. Examples of topical products commonly classified as “topical drugs” are many and varied, and include over-the-counter and/or prescription products such as antiperspirants, insect repellents, sunscreens and sunburn treatments, anti-acne agents, antibiotics, topical respiratory agents, ocular drugs such as eye drops and saline solutions, therapeutic retinoids, anti-dandruff agents, external analgesics such as capsaicin products, topical contraceptives, topical drug delivery systems, gastrointestinal agents such as suppositories, enemas and hemorrhoid treatments, reproductive system agents such as vaginal treatments, oral treatments such as lozenges, and many other products with therapeutic or other effects. Other topical products include hand, facial and body soaps and detergents and other forms of skin cleansers, as well as household detergents and many other household products such as solvents, propellants, polishes, lubricants, adhesives, waxes and others which are either applied topically or are topically exposed to the body during normal use.

It is of course known to the person skilled in the art that demanding cosmetic compositions are usually inconceivable without the customary auxiliaries and additives. Among these are included, for example, consistency-imparting agents, fillers, perfume, colorants, emulsifiers, additional active compounds such as vitamins or proteins, sunscreens, stabilizers, insect repellents, alcohol, water, salts, substances having antimicrobial, proteolytic or keratolytic activity, etc.

Suitable topical vehicles for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifing agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids (including dissolved forms of the inositol/IP-6 compositions of the invention as well as suspensions, emulsions and the like); solids and semisolids such as gels, foams, pastes, creams, ointments, “sticks” (as in lipsticks or underarm deodorant sticks), powders and the like; formulations containing liposomes or other delivery vesicles; rectal or vaginal suppositories, creams, foams, gels, ointments, enemas or douches; and other forms. Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or premoistened bandage, wipe, washcloth or stick—to the skin); spraying (including mist, aerosol or foam spraying); dropper application (as for example with ear or eye drops); sprinkling (as with a suitable powder form of the formulation); soaking; and injection (particularly intradermal or subcutaneous injection). Iontophoresis or other electromagnetic-enhanced delivery systems may also be usefully employed, as for example to increase delivery to the dermis.

Methodologies and materials for preparing formulations in a variety of forms are also described in Anthony L. L. Hunting (ed.), “A Formulary of Cosmetic Preparations (Vol. 2)—Creams, Lotions and Milks,” Nacelle Press (England, N.J. 1993). See, for example, Chapter 7, pp. 5-14 (oils and gels); Chapter 8, pp. 15-98 (bases and emulsions); Chapter 9, pp. 101-120 (“all-purpose products”); Chapter 10, pp. 121-184 (cleansing masks, creams, lotions); Chapter 11, pp. 185-208 (foundation, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 (baby products); the contents of which are incorporated herein by reference.

The corresponding requirements apply mutatis mutandis to the formulation of medicinal preparations.

Medicinal topical compositions within the meaning of the present invention as a rule contain one or more medicaments in an efficacious concentration. For the sake of simplicity, for clearer differentiation between cosmetic and medicinal use and appropriate products refer to the legal requirements of the United States (e.g. Cosmetics Regulations, Food and Drugs Act).

It is likewise advantageous in this case to add the active compound used according to the invention as an additive to preparations which already contain other active compounds for other purposes.

Accordingly, cosmetic or topical dermatological compositions within the meaning of the present invention can, for example, be used, depending on their composition, as skin protection cream, cleansing milk, sunscreen lotion, sun tan lotion, nourishing cream, day or night cream, etc. It is optionally possible and advantageous to use the compositions according to the invention as a base for pharmaceutical formulations.

In particular, the active compound used according to the invention can be used as an additive in cosmetic deodorants or antiperspirants. Agents having deodorant or antiperspirant activity which can be used are then the customary substances known to the person skilled in the art. For example, by means of astringents—mainly aluminum salts such as aluminum hydroxychloride—the formation of perspiration can be suppressed.

By the use of antimicrobial/biocidal substances in cosmetic deodorants, the bacterial flora on the skin can be reduced. At the same time, in the ideal case only the odor-causing microorganisms in the skin should be effectively reduced. Monocarboxylic acid esters of di- or triglycerol, for example, are advantageous. However, other substances having antimicrobial activity are also suitable.

Cosmetic and dermatological preparations also convenient for the purpose of the present invention are those in the form of a sunscreen. Besides the active compound used according to the invention, these preferably additionally contain at least one UVA filter substance and/or at least one UVB filter substance and/or at least one inorganic pigment.

However, it is also advantageous within the meaning of the present invention to make available those cosmetic and dermatological preparations whose main purpose is not protection from sunlight, but which nevertheless contain UV-protective substances. Thus UV-A and UV-B filter substances are usually incorporated, for example, in day creams.

Advantageously, preparations according to the invention contain substances which absorb UV radiation in the UVB range, the total amount of the filter substances being, for example, 0.1% by weight to 30% by weight, preferably 0.5 to 10% by weight, in particular 1 to 6% by weight, based on the total weight of the preparations.

The UVB filters can be oil-soluble or water-soluble. Oil-soluble substances which may be mentioned are, for example: 3-benzylidenecamphor and its derivatives, e.g. 3-(4-methylbenzylidene)camphor; 4-aminobenzoic acid derivatives, preferably 2-ethyl-hexyl4-dimethylaminobenzoate, amyl 4-dimethylaminobenzoate; esters of cinnamic acid, preferably 2-ethylhexyl4-methoxycinnamate, isopentyl 4-methoxycinnamate; esters of salicylic acid, preferably 2-ethylhexyl salicylate, 4-isopropylbenzyl salicylate, homomenthyl salicylate; derivatives of benzophenone, preferably 2-hydroxy-4-methoxybenzophenone, 2-hydroxy-4-methoxy-4′methylbenzophenone, 2,2′-dihydroxy-4-methoxybenzophenone; esters of benzylidenemalonic acid, preferably di(2-ethylhexyl) 4-methoxybenzylidenemalonates; 2,4,6-trianilino(p-carbo-2′-ethyl-1′-hexyloxy)-1,3,5-triazine

Advantageous water-soluble substances are: 2-phenylbenzimadazole-5-sulphonic acid and its salts, e.g. sodium, potassium or triethanolammonium salts, sulphonic acid derivatives of benzophenones, preferably 2-hydroxy-4-methoxybenzophenone-5-sulphonic acid and its salts; sulphonic acid derivatives of 3-benzylidenecamphor, such as, for example, 4-(2-oxo-3-bomylidenemethyl)-benzenesulphonic acid, 2-methyl-5-(2-oxo-3-bomylidnemethyl)sulphonic acid and their salts.

The list of UVB filters mentioned which can be used according to the invention is of course not intended to be limiting.

The invention also relates to the combination of inositol/IP-6 compositions with a UVA filter and a UVB filter or a cosmetic or dermatological preparation according to the invention which also contains a UVA filter and a UVB filter.

It may also be advantageous to employ in preparations according to the invention UVA filters which are customarily contained in cosmetic and/or dermatological preparations. Such filter substances are preferably derivatives of dibenzoylmethane, in particular 1-(4′-tert-butylphenol)-3-(4′-methoxyphenyl)propane-1,3-dione and 1-phenyl-3-(4′-isopropylphenyl)propane-1,3-dione. Preparations which contain these combinations are also a subject of the invention. The same amounts of UVA filter substances can be used which were mentioned for UVB filter substances.

Cosmetic and/or dermatological preparations within the meaning of the present invention can also contain inorganic pigments which are customarily used in cosmetics for the protection of the skin from UV rays. These are oxides of titanium, zinc, iron, zirconium, silicon, manganese, aluminum, cerium and mixtures thereof, as well as modifications in which the oxides are the active agents. They are particularly preferably pigments based on titanium dioxide. The amounts mentioned for the above combinations can be used.

The cosmetic and dermatological preparations according to the invention can contain cosmetic active compounds, auxiliaries and/or additives such as are customarily used in such preparations, e.g. antioxidants, preservatives, bactericides, perfumes, substances for preventing foaming, colorants, pigments which have a coloring action, thickeners, surface-active substances, emulsifiers, emollients, moisturizing and/or moisture-retaining substances, fats, oils, waxes or other customary constituents of a cosmetic or dermatological formulation such as alcohols, polyols, polymers, foam stabilizers, electrolytes, organic solvents or silicone derivatives.

It is advantageous to add to the preparations within the meaning of the present invention further anti-irritants or anti-inflammatory active compounds, in particular butyl alcohol (a-octadecyl glyceryl ether), selachyl alcohol (α-9-octadecenyl glyceryl ether), chimyl alcohol (α-hexadecyl glyceryl ether), bisabolol and/or panthenol.

It is also advantageous to add to the preparations within the meaning of the present invention customary antioxidants. According to the invention, convenient antioxidants which can be used are all antioxidants suitable or utilizable for cosmetic and/or dermatological applications.

Advantageously, the antioxidants are selected from the group consisting of amino acids (e.g. glycine, histidine, tyrosine, tryptophan) and their derivatives, imidazoles (e.g. urocanic acid) and its derivatives, peptides such as D,L-camosine, D-camosine, L-carnosine and their derivatives (e.g. anserine), carotenoids, carotenes (e.g. α-carotene, β-carotene, lycopene) and their derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and also their salts, dilauryl thiodipropionate, distearyl thiodipropionate, thiodipropionic acid and its derivatives (esters, ethers, peptides, lipids, nucleotides, nucleosides and salts) and also sulphoximine compounds (e.g. buthionine sulphoximine, homocysteine sulphoximine, buthionine sulphone, penta-, hexa- and heptathionine sulphoximine) in very low tolerable doses (e.g. pmol to μmol/kg), furthermore (metal) chelators (e.g. α-hydroxy fatty acids, palmitic acids, phytic acid, lactoferrin), α-hydroxy acids (e.g. citric acid, lactic acid, maleic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbyl palmitate, Mg-ascorbyl phosphate, ascorbyl acetate), tocopherols and derivatives (e.g. vitamin E acetate) and also coniferyl benzoate of gum benzoin, rutic acid and its derivatives, ferulic acid and its derivatives, butylhydroxytoluene, butylhydroxyanisole, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbene and its derivatives (e.g. stilbene oxide, trans-stilbene oxide) and the derivatives suitable according to the invention (salts, esters, ethers, sugars, nucleotides, nucleosides, peptides and lipids) of the active compounds mentioned.

The amount of the antioxidants (one or more compounds) in the preparations is preferably 0.001 to 30% by weight, particularly preferably 0.05-20% by weight, in particular 1-10% by weight, based on the total weight of the preparation.

If vitamin E and/or its derivatives is/are the antioxidant(s), it is advantageous to select the respective concentrations thereof from the range from 0.001 to 10% by weight, based on the total weight of the formulation.

If the cosmetic or dermatological preparation within the meaning of the present invention is a solution or emulsion or dispersion, the following can be used as solvents: water or aqueous solutions; oils, such as triglycerides of capric or of caprylic acid, but preferably castor oil; fats, waxes and other natural and synthetic fatty materials, preferably esters of fatty acids with alcohols of low C number, e.g. with isopropanol, propylene glycol or glycerol, or esters of fatty alcohols with alkanoic acids of low C number or with fatty acids; alcohols, diols or polyols of low C number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

The oil phase of the emulsions, oleogels or hydrodispersions or lipodispersions within the meaning of the present invention is advantageously selected from the group consisting of the esters of saturated and/or unsaturated, branched and/or unbranched alkanecarboxylic acids of a chain length of 3 to 30 C atoms and saturated and/or unsaturated, branched and/or unbranched alcohols of a chain length of 3 to 30 C atoms, from the group consisting of the esters of aromatic carboxylic acids and saturated and/or unsaturated, branched and/or unbranched alcohols of a chain length of 3 to 30 C atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and also synthetic, semisynthetic and natural mixtures of such esters, e.g. jojoba oil.

Furthermore, the oil phase can advantageously be selected from the group consisting of the branched and unbranched hydrocarbons and hydrocarbon waxes, the silicone oils, the dialkyl ethers, the group consisting of the saturated or unsaturated, branched or unbranched alcohols, and also the fatty acid triglycerides, namely the triglycerol esters of saturated and/or unsaturated, branched and/or unbranched alkanecarboxylic acids of a chain length of 8 to 24, in particular 12-18, C atoms. The fatty acid triglycerides can, for example, be advantageously selected from the group consisting of the synthetic, semisynthetic and natural oils, e.g. olive oil, sunflower oil, soya bean oil, ground nut oil, rape seed oil, almond oil, palm oil, coconut oil, palm kernel oil and the like.

### Anti-Inflammatory Compositions

In an aspect, inositol/IP-6 compositions of the present invention can be formulated with an anti-inflammatory agent in a cosmetic base or dental linament (periodontal disease) for topical application for local prevention of inflammation and/or tissue damage consequent to inflammation. A variety of steroidal and non-steroidal anti-inflammatory agents can be combined with inositol/IP-6 compounds.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, flupreclnisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use in the present invention is hydrocortisone.

Specific non-steroidal anti-inflammatory agents useful in the composition of the present invention include, but are not limited to: piroxicam, isoxicam, tenoxicam, sudoxicam, CP-14,304, aspirin, disalcid, benorylate, trilisate, safapryn, solprin, diflunisal, fendosal, diclofenac, fenclofenac, indomethacin, sulindac, tolmetin, isoxepac, furofenac, tiopinac, zidometacin, acemetacin, fentiazac, zomepirac, clidanac, oxepinac, felbinac, mefenamic, meclofenamic, flufenamic, niflumic, tolfenamic acids, ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, tiaprofenic, phenylbutazone, oxyphenbutazone, feprazone, azapropazone, and trimethazone, among others. Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamic acid, meclofenamic acid, piroxicam and felbinac are preferred and ibuprofen, naproxen, and flufenamic acid are most preferred.

Finally, so-called “natural” anti-inflammatory agents are useful in the present invention. For example, candelilla wax, alpha bisabolol, aloe vera, Manjistha (extracted from plants in the genus Rubia, particularly Rubia Cordifolia), and Guggul (extracted from plants in the genus Commiphora, particularly Commiphora Mukul), may be used.

While aqueous solvents are generally preferred, the pharmaceutical/cosmetic compositions of the present invention formulated as solutions may include a pharmaceutically- or cosmetically-acceptable organic solvent. The terms “pharmaceutically-acceptable organic solvent” and “cosmetically-acceptable organic solvent” refer to an organic solvent which, in addition to being capable of having dispersed or dissolved therein the inositol/IP-6 compound, and optionally also an anti-inflammatory or other agent, also possesses acceptable safety (e.g. irritation and sensitization characteristics), as well as good aesthetic properties (e.g., does not feel greasy or tacky). The most typical example of such a solvent is isopropanol. Examples of other suitable organic solvents include: propylene glycol, polyethylene glycol (200-600), polypropylene glycol (425-2025), glycerol, 1,2,4-butanetriol, sorbitol esters, 1,2,6-hexanetriol, ethanol, butanediol, water and mixtures thereof. These solutions contain from about 0.01% to about 5%, preferably from about 0.5% to about 2% of an anti-inflammatory agent.

As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials. Examples of classes of useful emollients include the following: hydrocarbon oils and waxes, including mineral oil, petrolatum, paraffin, ceresin, ozokerite, microcrystalline wax, polyethylene, and perhydrosqualene; silicone oils, such as dimethyl polysiloxanes, methylphenyl polysiloxanes, water-soluble and alcohol-soluble silicone glycol copolymers; triglyceride esters, for example vegetable and animal fats and oils, including castor oil, safflower oil, cottonseed oil, corn oil, olive oil, cod liver oil, almond oil, avocado oil, palm oil, sesame oil, and soybean oil; acetoglyceride esters, such as acetylated monoglycerides; ethoxylated glycerides, such as ethoxylated glyceryl monostearate; alkyl esters of fatty acids having 10 to 20 carbon atoms, such as methyl, isopropyl, and butyl esters of fatty acids, hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, auryl lactate, myristyl lactate, and cetyl lactate; alkenyl esters of fatty acids having 10 to 20 carbon atoms, including oleyl myristate, oleyl stearate, and oleyl oleate; fatty acids having 10 to 20 carbon atoms such as pelargonic, lauric, myristic, palmitic, stearic, isostearic, hydroxystearic, oleic, linoleic, ricinoleic, arachidic, behenic, and erucic acids; fatty alcohols having 10 to 20 carbon atoms such as lauryl, myristyl, cetyl, hexadecyl, stearyl, isostearyl, hydroxystearyl, oleyl, ricinoleyl, behenyl, and erucyl alcohols, as well as 2-octyl dodecanol; fatty alcohol ethers such as ethoxylated fatty alcohols of 10 to 20 carbon atoms, including the lauryl, cetyl, stearyl, isostearyl, oelyl, and cholesterol alcohols having attached thereto from 1 to 50 ethylene oxide groups or 1 to 50 propylene oxide groups; ether-esters such as fatty acid esters of ethoxylated fatty alcohols; lanolin and derivatives, such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; polyhydric alcohols and polyether derivatives such as propylene glycol, dipropylene glycol, polypropylene glycols 2,000 and 4,000, polyoxyethylene polyoxypropylene glycols, polyoxypropylene polyoxyethylene glycols, glycerol, sorbitol, ethoxylated sorbitol, hydroxypropyl sorbitol, polyethylene glycols 200-6,000, methoxy polyethylene glycols 350, 550, 750, 2,000 and 5,000, poly[ethylene oxide]homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C15-C18 vicinal glycol, and polyoxypropylene derivatives of trimethylolpropane; polyhydric alcohol esters, including ethylene glycol mono- and di-fatty acid esters, diethylene glycol mono- and di-fatty acid esters, polyethylene glycol (200-6,000) mono- and di-fatty acid esters, propylene glycol mono- and di-fatty acid esters, polypropylene glycol 2,000 monooleate, polypropylene glycol 2,000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters; wax esters such as beeswax, spermaceti, myristyl myristate, stearyl stearate; beeswax derivatives, e.g. polyoxyethylene sorbitol beeswax which are reaction products of beeswax with ethoxylated sorbitol of varying ethylene oxide content, forming a mixture of ether-esters; vegetable waxes including carnauba and candelilla waxes; phospholipids, such as lecithin and derivative; sterols, such as cholesterol and cholesterol fatty acid esters; and amides such as fatty acid amides, ethoxylated fatty acid amides, solid fatty acid alkanolamides.

Particularly useful emollients which provide skin conditioning are glycerol, hexanetriol, butanetriol, lactic acid and its salts, urea, pyrrolidone carboxylic acid and its salts, amino acids, guanidine, diglycerol and triglycerol. Preferred skin conditioning agents are the propoxylated glycerol derivatives.

The compositions of the present invention may also be delivered by spray. Suitable propellants for cosmetic and/or dermatological preparations within the meaning of the present invention, which can be sprayed from aerosol containers are the customary known easily volatile, liquefied propellants, for example hydrocarbons (propane, butane, isobutane), which can be employed on their own or as a mixture. Compressed air can also be advantageously used.

Of course, the person skilled in the art knows that there are non-toxic propellant gases which would be fundamentally suitable for the realization of the present invention in the form of aerosol preparations, but which nevertheless should be dispensed with because of dubious effects on the environment or other concomitant circumstances, in particular fluorohydrocarbons and chlorofluorocarbons (CFCs).

### Preservatives

The inositol/IP-6 compounds of the present invention can be used as preservatives in perishable items such as foods and pharmaceuticals and to prevent fungal growth on the surface of fresh fruits or may include such preservatives in their compositions. Presently, chemicals such as citrashine orthophenilphenol thiabendazole are used. Stability experiments show that inositol/IP-6 compounds, for example, are highly stable when used as a preservative in foods and on the surface of fresh fruits. Microbial and fungal growth is inhibited while the food components are unaffected. The preservative of the present invention has less likelihood of toxicity or untoward reactions if ingested that the present, complex chemical antifungals. The preferred concentration of inositol/IP-6 or acceptable salt or derivative for this use is approximately below 0.025% to be classified as a preservative. Of course, greater percentages, e.g. up to 99.9% also would be effective.

### Zinc-Finger and Iron-Finger Hormone Receptor Proteins and Aging and Carcinogenesis

At physiological concentrations, transition metal ions, such as iron, cobalt and copper are essential elements for biological functions; at higher levels, however, they are toxic. This is particularly true for iron. Toxicity of the transition metal ions, particularly iron, is the fact that protein domains are present within key enzyme and transcriptional regulatory molecules (DNA-binding proteins) which normally bind zinc (zinc finger domains) but which can substitute zinc by other transition metals that are present in the cell. Elevated levels of iron contribute to carcinogenesis in several ways; iron has the capacity to generate highly reactive free radicals that damage DNA, and rapidly proliferating transformed cells have increased requirement for iron for DNA replication (ribonucleotide reductase) and for energy production by mitochondria.

Iron can replace zinc in the zinc-containing hormone-receptor proteins for testosterone, progesterone and other hormones. Iron may also generate free radicals which damage DNA in specific regulatory regions and potentially induce carcinogenesis in the prostate, uterus, and other organs. Thus, classical hormones can modulate iron-finger receptor proteins. The hormones potentiate the destructive actions of free radicals, mediated by abnormal iron-finger receptor proteins, on regulatory regions of DNA. It is feasible to maintain zinc-finger proteins in an undamaged zinc-containing form by using a combination of specific agents, such as iron chelators and radical scavengers, respectively, to interfere with the formation of both aberrant iron-finger proteins and free radicals. Thus, inositol/IP-6 compositions and pharmacologically acceptable derivatives and salts thereof, in the dosages discussed above, can be used to prevent the formation of aberrant iron-finger proteins involved in carcinogenesis and aging.

### EXAMPLE 15 (PROPHETIC)

### Topical or Intravaginal Preparation of Inositol/IP-6 in an Absorption Base

A topical or intravaginal preparation of inositol/IP-6 in an absorption base is made by incorporating 0.001% to 99.9%, preferably 1% to 50%, most preferably 5% to 20% inositol/IP-6 into an absorption base. An absorption base generally is an anhydrous base which has the property of absorbing several times its weight of water to form an emulsion and still retain an ointment-like consistency. Absorption bases may vary in their composition but generally are a mixture of animal sterols with petrolatum, such as Hydrophilic Petrolatum, U.S.P. The most common commercially available products are Eucerin and Aquaphor (Beiersdorf) and Polysorb (Fougera). One preferred embodiment of the topical preparation is made by dissolving 10% inositol/IP-6 compounds in deionized water and then incorporating the solution into an equal amount of Aquaphor, on a wt/wt basis. Further, the inositol/IP-6 compounds or derivatives thereof can be incorporated into a balm or stick for application to the lips to treat herpes infections. It will be appreciated that inositol/IP-6 derivatives can be used in place of the inositol/IP-6 in the topical preparation. It will be appreciated that an appropriate concentration of a substituted inositol/IP-6 derivative can be used in place of the inositol/IP-6 without departing from the scope of the invention. It will be appreciated that such preparations can be used to treat topical conditions such as virus infections, fungal infections, susceptible bacterial infections, radiation assault, including ultraviolet, medical or atomic radiation, skin cancers or any other condition mediation by the above described mechanisms.

### EXAMPLE 15 (PROPHETIC)

### Acne Formulation and Sunburn Treatment

A preparation useful in the treatment and control of acne comprises approximately 7.5% to about 10% inositol/IP-6 compounds, by weight, in a suitable topical lotion. The acne preparation can include approximately 1% to approximately 99% inositol/IP-6 compounds, derivatives or analogs thereof. Preferably, the composition will also include other acne medications such as retinoid derivatives. A preferred range is approximately 5% to approximately 15%. The lotion is applied to the skin two or three times daily.

The above described lotion also can be used to control the symptoms of sunburn.

### EXAMPLE 16 (PROPHETIC)

### Systemic Administration

A systemic preparation of inositol/IP-6 compounds containing approximately 1% to 100% active ingredient may be administered orally, intravenously or by any acceptable route. For example, inositol/IP-6 compositions prepared in 00 gelatin capsules at 1,250 mg per capsule has been shown to be effective in the prevention and/or amelioration of the adverse health effects of ionizing radiation exposure. The preparation can be provided as a flavored or unflavored oral solution. Likewise, an injectable form may be prepared.

As set out above, the safe and effective daily systemic dose may range for 750 mg to 5 grams for a 70 Kg subject, with the preferred range being 1 to 4 grams, and the most preferred dose being 1.5 grams to 2.5 grams.

### Kits

Kits can also be supplied for use with the subject inositol/IP-6 compositions for use in the protection against or therapy for exposure to ionizing radiation. Thus, the subject composition of the present invention may be provided, usually in a lyophilized form (powder or capsule), tablet or chewable tablet, or aqueous solution in a container, either alone or in conjunction with additional inositol/IP-6 compositions of the desired type. The inositol/IP-6 compositions are preferably included in the kits in unit doses of both an oral and topical formulation along with a set of instructions for use. Frequently, it will be desirable to include an inert extender or excipient to dilute the active ingredients, where the excipient may be present in from about 1 to 99.999% wt. of the total composition. The kits may optionally include, either separately or as a component of the oral or topical formulation, one or more of a biocide, nutritional supplement, sun block or sun screen, iodine tablets, a cosmetic colorant or paint, analgesic, pre-moistened towlettes, antiseptic ointment or wipes, or a food item. The packaging for the kit and its components is preferably disposable, and even more preferably biodegradable.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows:

1. A method for preventing or treating acute short-term adverse health effects of ionizing radiation exposure in a mammal, comprising:
administering to the mammal an effective amount of a pharmaceutical composition comprising IP-6, its pharmaceutically acceptable salts, or its pharmaceutically acceptable derivatives, in any combination; and
preventing or treating acute short-term adverse health effects of ionizing radiation exposure in a mammal.

2. The method of claim 1, wherein the pharmaceutical composition further comprises inositol.

3. The method of claim 1, wherein the pharmaceutical composition further comprises at least one pharmaceutically acceptable excipient or carrier.

4. The method of claim 1, wherein the pharmaceutical composition is a liquid, lotion, cream, gel, ointment, powder, tablet, chewable tablet, suppository, or capsule.

5. The method of claim 1, wherein the pharmaceutical composition is an enteral formulation.

6. The method of claim 5, wherein the pharmaceutical composition contains IP-6, its pharmaceutically acceptable salts, or its pharmaceutically acceptable derivatives, in any combination, in an amount from about 0.1% to about 100% by weight.

7. The method of claim 5, wherein the pharmaceutical composition contains IP-6, its pharmaceutically acceptable salts, or its pharmaceutically acceptable derivatives, in any combination, in an amount from about 0.1% to about 50% by weight, and further comprises inositol, its pharmaceutically acceptable salts, or its pharmaceutically acceptable derivatives, in any combination, in an amount from about 0.1% to about 50% by weight.

8. The method of claim 7, wherein the pharmaceutical composition contains inositol and IP-6, their pharmaceutically acceptable salts, or their pharmaceutically acceptable derivatives, in any combination, in a ratio of about 30:1 to about 1:30.

9. The method of claim 8, wherein the inositol and IP-6, their pharmaceutically acceptable salts, or their pharmaceutically acceptable derivatives, in any combination, are present in a ratio of about 5:1 to about 1:5.

10. The method of claim 1, wherein the pharmaceutical composition is a parenteral formulation.

11. The method of claim 10, wherein the pharmaceutical composition contains IP-6, its pharmaceutically acceptable salts, or its pharmaceutically acceptable derivatives, in any combination, in an amount from about 0.01% to about 20% by weight.

12. The method of claim 10, wherein the pharmaceutical composition contains IP-6, its pharmaceutically acceptable salts, or its pharmaceutically acceptable derivatives, in any combination, in an amount from about 0.01% to about 20% by weight, and further comprises inositol, its pharmaceutically acceptable salts, or its pharmaceutically acceptable derivatives, in any combination, in an amount from about 0.01% to about 20% by weight.

13. The method of claim 10, wherein the pharmaceutical composition contains IP-6, its pharmaceutically acceptable salts, or its pharmaceutically acceptable derivatives, in any combination, in an amount from about 0.1% to about 10% by weight, and further comprises inositol, its pharmaceutically acceptable salts, or its pharmaceutically acceptable derivatives, in any combination, in an amount from about 0.1% to about 10% by weight.

14. The method of claim 10, wherein the pharmaceutical composition contains IP-6, its pharmaceutically acceptable salts, or its pharmaceutically acceptable derivatives, in any combination, in an amount from about 0.5% to about 5% by weight, and further comprises inositol, its pharmaceutically acceptable salts, or its pharmaceutically acceptable derivatives, in any combination, in an amount from about 0.5% to about 5% by weight.

15. The method of claim 12, wherein the pharmaceutical composition contains inositol and IP-6, their pharmaceutically acceptable salts, or their pharmaceutically acceptable derivatives, in any combination, in a ratio of about 30:1 to about 1:30.

16. The method of claim 15, wherein the inositol and IP-6, their pharmaceutically acceptable salts, or their pharmaceutically acceptable derivatives, in any combination, are present in a ratio of about 5:1 to about 1:5.

17. The method of claim 1, wherein the pharmaceutical composition further comprises an antioxidant, a biocide, a chemotherapeutic, a nutritional supplement or nutraceutical, an analgesic, a sunblock, a moisturizer, or any combination thereof.

18. The method of claim 1, wherein the inositol and IP-6 are present in the forms of pharmaceutically acceptable salts, isomers, esters, derivatives, or any combination thereof.

19. The method of claim 1, wherein the pharmaceutical composition is administered for at least one day prior to ionizing radiation exposure.

20. The method of claim 19, wherein the administration is performed at least twice daily.

21. The method of claim 19, wherein the administration is performed at least three times daily.

22. The method of claim 5, wherein the pharmaceutical composition is administered in at least one dose containing a total of about 1 gram to about 10 grams of inositol, IP-6, their pharmaceutically acceptable salts, or their pharmaceutically acceptable derivatives, in any combination.

23. The method of claim 5, wherein the pharmaceutical composition is administered in at least one dose containing about 2 gram to about 5 grams of inositol, IP-6, their pharmaceutically acceptable salts, or their pharmaceutically acceptable derivatives, in any combination.

24. The method of claim 5, wherein the pharmaceutical composition is administered orally as a powder, tablet, or capsule and topically as a lotion, cream, ointment or gel.

25. The method of claim 1, wherein the ionizing radiation exposure comprises ultraviolet light, x-rays, gamma rays, cosmic rays, particle beams, or any combination thereof.

26. The method of claim 1, wherein the ionizing radiation derives from one or more natural sources.

27. The method of claim 26 wherein the one or more natural sources comprise: the sun, outer space, or radioactive elements present in the atmosphere, ground, mineral deposits, mined ore, groundwater, bodies of water, or stone.

28. The method of claim 1, wherein the ionizing radiation derives from one or more human-derived sources.

29. The method of claim 28, wherein the human-derived sources comprise: ultraviolet lights, therapeutic radiation sources, nuclear power plants, nuclear fuel, nuclear weapons, nuclear fallout, and radioactive consumer devices.

30. The method of claim 1, wherein the adverse health effects comprise: skin burns, rashes, mucosal degradation or bleeding, gastro-intestinal degradation or bleeding, diarrhea, anemia, or excessive fatigue.

31. A method for safely increasing the dosage of therapeutic ionizing radiation provided to a mammal in need of ionizing radiation therapy, comprising:
Administering to a mammal prior to exposure to therapeutic radiation ionizing radiation a pharmaceutical composition comprising a composition according to claim 1; and
Exposing the mammal to a dosage of therapeutic radiation ionizing radiation greater than a maximum safe dosage for said mammal in the absence of said pharmaceutical composition.

32. A method for protecting a worker from short-term adverse health effects of workplace ionizing radiation exposure, comprising:
Administering to a worker prior to exposure to workplace ionizing radiation a pharmaceutical composition comprising a composition according to claim 1; and
protecting the worker from adverse health effects of workplace ionizing radiation exposure.

33. A method for protecting military personnel from short-term adverse health effects of human-derived ionizing radiation exposure, comprising:
Administering to military personnel prior to exposure to military ionizing radiation a pharmaceutical composition comprising a composition according to claim 1; and
protecting the military personnel from adverse health effects of human-derived ionizing radiation exposure.

34. A kit used for protecting military personnel from short-term adverse health effects of human-derived ionizing radiation exposure, comprising:
A container, containing a plurality of pharmaceutical compositions comprising IP-6, its pharmaceutically acceptable salts, or its pharmaceutically acceptable derivatives, in any combination, said plurality of pharmaceutical compositions comprising a topical preparation and an oral preparation effective to protect military personnel from short-term adverse health effects of human-derived ionizing radiation exposure.

35. The kit of claim 34, wherein the plurality of pharmaceutical compositions are provided in unit doses.

36. The kit of claim 34, containing sufficient unit doses for at least one day's usage.

37. The kit of claim 34, wherein at least one of the plurality of pharmaceutical compositions further comprises inositol, its pharmaceutically acceptable salts, or its pharmaceutically acceptable derivatives, in any combination.

38. The kit of claim 37, wherein the plurality of pharmaceutical compositions are administered in at least one dose each containing a total of about 1 gram to about 10 grams of inositol, IP-6, their pharmaceutically acceptable salts, or their pharmaceutically acceptable derivatives, in any combination.

39. A topical preparation for preventing acute short-term adverse health effects of ionizing radiation exposure in a mammal, comprising:
an effective amount of a composition comprising IP-6, its pharmacologically acceptable salts, or its pharmacologically acceptable derivatives, in any combination, and at least one pharmacologically acceptable carrier, effective to prevent acute short-term adverse health effects of ionizing radiation exposure in a mammal.

40. The topical preparation of claim 39, wherein the composition is a lotion, cream, or gel.

41. The topical preparation of claim 39, wherein the acute short-term adverse health effects of ionizing radiation exposure comprise sunburn.

42. The topical preparation of claim 39, wherein the composition is applied to the skin of the mammal at a time sufficiently prior to the ionizing radiation exposure to allow the IP-6, its pharmacologically acceptable salts, or its pharmacologically acceptable derivatives, in any combination, to be absorbed by cells of the skin.

43. The topical preparation of claim 39, wherein the pharmaceutical composition is applied to the skin of the mammal three to twelve hours prior to the ionizing radiation exposure.

44. The topical preparation of claim 39, further comprising an antioxidant, a biocide, a nutritional supplement or nutraceutical, an analgesic, a sunblock, a sun tanning preparation, a moisturizer, or any combination thereof.

45. The topical preparation of claim 39, further comprising inositol, its pharmaceutically acceptable salts, or its pharmaceutically acceptable derivatives, in any combination.

46. The topical preparation of claim 40, wherein the composition contains IP-6, its pharmaceutically acceptable salts, or its pharmaceutically acceptable derivatives, in any combination, in an amount from about 0.1% to about 50% by weight, and further comprises inositol, its pharmaceutically acceptable salts, or its pharmaceutically acceptable derivatives, in any combination, in an amount from about 0.1% to about 50% by weight.

47. The topical preparation of claim 46, wherein the composition contains inositol and IP-6, their pharmaceutically acceptable salts, or their pharmaceutically acceptable derivatives, in any combination, in a ratio of about 30:1 to about 1:30.

48. The topical preparation of claim 46, wherein the inositol and IP-6, their pharmaceutically acceptable salts, or their pharmaceutically acceptable derivatives, in any combination, are present in a ratio of about 5:1 to about 1:5.
